(19) United States
(12) Patent Application Publication
(10) Pub. No.: US 2007/0122395 A1
(43) Pub. Date: May 31, 2007

(54) Genetic and pharmacological regulation of antidepressant-sensitive biogenic amine transporters through PKG/p38 map kinase

(75) Inventors: Randy D. Blakely, Brentwood, TN (US); Chong-Bin Zhu, Nashville, TN (US); Harish C. Prasad, Nashville, TN (US); William A. Hewlett, Nashville, TN (US)

Correspondence Address: FULBRIGHT & JAWORSKI L.L.P., 600 CONGRESS AVE., SUITE 2400, AUSTIN, TX, 78701, US

(73) Assignee: Vanderbilt University

(21) Appl. No.: 11/545,023

(22) Filed: Oct. 6, 2006

Related U.S. Application Data
(60) Provisional application No. 60/727,077, filed on Oct. 14, 2005.
(60) Provisional application No. 60/724,065, filed on Oct. 6, 2005.

(51) Int. Cl. A61K 48/00; C12Q 1/68; G01N 33/567

(52) U.S. Cl. 424/93.21; 514/2; 514/44; 435/6; 435/7.2

(57) ABSTRACT

The invention relates to the observation that the p38 mitogen-activated protein kinase pathway is an important regulator of biogenic amine transporter (BAT) function. By using modulator of p38 MAPK, one can alter BAT function in a specific manner. This recognition of the pathway and its interaction with the serotonin transporter (SERT) and the norepinephrine transporter (NET), along with the PPA, PKG and PDE pathways, also provides the opportunity to identify polymorphisms that will impact the efficacy of drugs that act though on these enzymes or their pathways.

The present application claims benefit of priority to U.S. Provisional Application Ser. Nos. 60/727,070 and 60/724,065, filed Oct. 14, 2005 and Oct. 5, 2005, respectively. The entire contents of both of these applications are hereby incorporated by reference.

The government owns rights in the present invention pursuant to grant numbers R01DA07390 from the National Institutes of Health.

### BACKGROUND OF THE INVENTION

1. Field of the Invention

The present invention relates generally to the fields of neurobiology, pharmacology and psychiatry. More particularly, it concerns a method for altering SERT activity and identifying variants of SERT that affect drug activity.

2. Description of Related Art

A. Serotonin Transporter

Neurotransmitters mediate signal transduction in the nervous system and modulate the processing of responses to a variety of sensory and physiological stimuli. An important regulatory step in neurotransmission is the inactivation of a neurotransmitter following its release into the synaptic cleft. This is especially true for the biogenic amine and amino acid neurotransmitters. Inactivation of neurotransmitters is typically mediated by uptake of the released neurotransmitter by neurotransmitters transporters that are located on the presynaptic neuron or in some cases on adjacent glial cells. Thus, neurotransmitter transporters are central to the processing of information in the nervous system and are associated with numerous neurological disorders.

Serotonin and the serotonin transporter mediate diverse aspects of neuronal signaling and are involved in the pathology of a number of nervous system related disorders. The serotonin transporter (SERT) is a target of various therapeutic agents used in the treatment of neurological and neurodegenerative disorders. However, there continues to be a need for new drugs that target SERT with different activity profiles. In addition, it is important to identify alterations in SERT that impact the way that drugs will affect various individuals with differing genotypes.

Serotonin (5-hydroxytryptamine, 5-HT) is a neurotransmitter in the brain and periphery, and modulates a wide variety of physiological processes including vasoconstriction, gastrointestinal motility and secretion, respiration, sleep, appetite, aggression, and mood (Jacobs and Azmitia, 1992; Fozzard, 1989). Disrupted 5-HT signaling has been implicated in a similarly wide-spectrum of disorders including primary pulmonary hypertension, irritable bowel syndrome, sudden infant death syndrome (SIDS), anorexia, obsessive-compulsive disorder (OCD), autism, depression and suicide (Insel et al., 1990; Melzter, 1990; Gershon, 1999; Cook and Leventhal, 1996). A major determinant of 5-HT signaling is the antidepressant-sensitive 5-HT transporter (SERT, 5HTT). Human SERT (hSERT) protein is encoded by a single locus mapping to chromosome 17q 1.2 (Ramamoorthy et al., 1993). Although evidence of alternative splicing of 5′ non-coding exons exists (Bradley and Blakely, 1997; Ozsarac et al., 2002), the same open reading frame is translated in brain, platelets, lymphocytes and placenta, producing a protein of 630 amino acids with closest identify to norepinephrine and dopamine transporters (NET and DAT respectively). Initial hydropathy-based predictions of SERT secondary structure proposed 12 transmembrane domains (TMs) with intracellular NH2 and COOH termini (Hoffman et al., 1991), a model supported by biochemical and immunocytochemical studies (Chen et al., 1998; Miner et al., 2000).

B. SERT Regulation

SERT proteins can be rapidly regulated by multiple G-protein coupled receptors and protein kinase-linked pathways, including those triggered by activation of protein kinase C (PKC) and protein kinase G (PKG) (Ramamoorthy et al., 1998; Ramamoorthy and Blakely, 1999; Zhu et al., 2004a,b; Zhu et al., 2005). Phosphorylation and downregulation of SERT through the PKC-linked pathway is sensitive to extracellular 5-HT (Ramamoorthy and Blakely, 1999), revealing an intrinsic capacity for temporal integration of ongoing 5-HT clearance demand with modulatory inputs. There is also anecdotal evidence of the involvement of the p38 mitogen-activated protein kinase (MAPK) pathway in regulating SERT activity. Zhu et al. (2003) reported that an inhibitor of p38 MAPK could block induction of 5-HT uptake caused by NECA, hydroxylamine and 8-br-cGMP. Zhu et al. (2004a) showed than anisomycin, a p38 MAPK activator, stimulated 5-HT uptake in platelets, and that a p38 MAPK inhibitor blocked H₂O₂- and UV light-activated 5-HT uptake. This work was extended by showing that a complicated series of pathways, including p38 MAPK and PKG, regulate 5-HT transport (Zhu et al., 2004b). However, these reports did not show that p38 MAPK activation directly triggered SERT enhancements, but only that p38 MAPK inhibitors blocked regulation by an adenosine receptor. The ability to use modulators of p38 MAPK to affect SERT-associated disease states has yet to be explored.

C. SERT Variants

SERT proteins can be rapidly regulated by multiple G-protein coupled receptors and protein kinase-linked pathways, including those triggered by activation of protein kinase C (PKC), protein kinase G (PKG) and possibly p38 mitogen activated protein kinase (MAPK) (Ramamoorthy et al., 1998; Ramamoorthy and Blakely, 1999; Zhu et al, 2004a,b; Samuvel et al., 2005; Zhu et al., 2005). Phosphorylation and downregulation of SERT through the PKC-linked pathway is sensitive to extracellular 5-HT (Ramamoorthy and Blakely, 1999), revealing an intrinsic capacity for temporal integration of ongoing 5-HT clearance demand with modulatory inputs. The importance of SERT in presynaptic 5-HT homeostasis, synaptic 5-HT clearance and psychoactive drug action has raised questions as to whether the hSERT gene exhibits functional polymorphisms that impact expression and activity in vivo (Murphy et al., 2004).

A common promoter variant (5HTTLPR) has been found to support altered hSERT mRNA and protein expression (Lesch et al., 1996) and has been associated with anxiety traits as well as multiple psychiatric syndromes including autism, OCD and depression (Murphy et al., 2004). A variable nucleotide tandem repeat sequence (VNTR) in the intron following the first coding exon has also been described and appears to have enhancer-like properties (MacKenzie and Quinn, 1999). Ten nonsynonymous single nucleotide polymorphisms (SNPs) have been identified in hSERT (Glatt et al., 2001; Di Bella et al., 1996), though few have been explored for their functional impact. Recently, Kilic and coworkers established a gain-of-function phenotype associated with the hSERT Ile425Val variant (Kilic et al., 2003), attributing alterations to constitutive elaboration of regulation normally supported by PKG stimulation. Ozaki and coworkers found the variant in two families, tracking the allele (as well as the 5HTTLPR “L” allele) with subjects exhibiting a complex psychiatric phenotype including, among other things, OCD and Asperger's Syndrome (Ozaki et al., 2003). However, a definitive analysis of SERT variants and their ability to affected by various intracellular signaling pathways has yet to be performed.

### SUMMARY OF THE INVENTION

Thus, in accordance with the present invention, there is provided a method of modulating anti-depressent biogenic amine transporter (BAT) function comprising contacting a BAT-expressing cell with a modulator of the p38 mitogen-activated protein kinase (MAPK) pathway and at least one other BAT modulator. The may be the serotonin transporter (SERT) or the norepinephrine transporter (NET). The other modulator may be a peptide, a polypeptide, a nucleic acid or an organopharmaceutical. The SERT or NET modulator may be an enhancer or inhibitor. The p38 MAPK modulator may be an agonist or antagonist. The p38 MAPK modulator may be a PPA2, PKG or PDE-5 modulator. The BAT-expressing cell may be a primary neuronal cell, an immortalized neuronal cell, or a cell recombinantly engineered to express a BAT.

In another embodiment, there is provided a method of modulating BAT function in a subject comprising administering to said subject a modulator of the p38 mitogen-activated protein kinase (MAPK) pathway. The cell may further be contacted with a BAT modulator. The p38 MAPK modulator may be an agonist or antagonist. The p38 MAPK modulator may be a PPA2, PKG or PDE-5 modulator. The BAT may be SERT or NET. The SERT or NET modulator may be an agonist or antagonist. The subject may suffer from anxiety, a stress disorder, depression, autism, anorexia, alcoholism, hypertension, irritable bowel syndrome, schizophrenia, obsessive compulsive disorder, primary pulmonary hypertension, migraine, autism, or premenstrual syndrome.

In yet another embodiment, there is provided a method of assessing drug sensitivity in a subject comprising determining the structure of a serotonin transporter in (SERT) cells of said subject. The step of determining the structure may comprise one or more of sequencing, Southern blot, Northern blot, Western blot, SNP analysis, RFLP, or primer extension. The step of determining the structure may also comprises one or more of: determining SERT protein mass, determining SERT phosphorylation state, determining SERT glycosylation state, or determining SERT 5HT flux/surface density ratios. The step of determining may comprise assessing for one or more of the following amino acid substitutions Thr4Ala, Gly56Ala, Glu215Lys, Lys605Asn, Pro612Ser, and Ile425Val.

The method may further comprise making a drug selection or drug dosing decision based on the structure of the SERT, and optionally, administering a selected drug to said subject. The drug may be a PKG or PDE-5 modulator, or a p38 mitogen-activated protein kinase pathway (MAPK) modulator, such as PPA1. The p38 MAPK modulator may be an agonist or antagonist. The drug may be a SERT modulator (enhancer or inhibitor). The subject may suffer from anxiety, a stress disorder, depression, autism, anorexia, alcoholism, hypertension, irritable bowel syndrome, schizophrenia, obsessive compulsive disorder, primary pulmonary hypertension, migraine, autism, or premenstrual syndrome. The subject may be a mammal such as a human.

It is contemplated that any method or composition described herein can be implemented with respect to any other method or composition described herein.

The use of the word, “a” or “an” when used with the term “comprising” in the specification and/or claims may mean “one,” “one or more,” “at least one,” or “one or more than one.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B—Location and 5-HT transport activity of human SERT coding variants. (FIG. 1A) Variants are overlayed on a 12 TM model of a single SERT subunit, with NH2 and COOH termini oriented inside the cell. Variants in extramembrane domains are shaded black whereas those in membrane domains are shaded white. (FIG. 1B) 5-HT transport activity of SERT coding variants in transfected HeLa cells. Data reflect mean values±SEM of 3 separate experiments. Means were compared to hSERT cDNA using a One-Way ANOVA followed by Dunnett's test of individual means against hSERT values with p<0.05 (*) taken as significant.

FIGS. 2A-C—Analysis of protein expression of hSERT and coding variants. (FIG. 2A) Immunoblots of total cell extracts prepared from HeLa cells transfected with hSERT or one of the variants described in the study. (FIG. 2B) Cell surface expression alterations in hSERT Pro339Leu and Ile425Val. Variants were transfected in parallel with hSERT into HeLa cells and cell surface transporters identified by immunoblotting of biotinylated samples, captured as described in Methods. (FIG. 2C) Quantitative estimations of relative surface density of hSERT, Pro339Leu and Ile425Val were based on densitometry of biotinylation immunoblots. Data reflect mean values of three separate experiments±SEM. Means were compared with a One-Way ANOVA followed by Dunnett's test to compare variant surface expression to that achieved with hSERT, with p<0.05 (*) taken as significant.

FIGS. 3A-B—Impact of 8BrcGMP and p38 MAPK on hSERT activity. (FIG. 3A) Activity modulation. HeLa cells transfected with hSERT or hSERT coding variants were examined for 5-HT transport activities as described in Methods following pretreatments of cells with either 100 μM 8BrcGMP+/−H8 or vehicle for 1 hr. (FIG. 3B) Altered p38 MAPK-dependent regulation of hSERT in transfected HeLa cells. Cells transfected with, hSERT or hSERT coding variants were examined following pretreatments of cells with either 1 μM anisomycin+/−SB203580 or vehicle for 10 min. Results reflect mean values±SEM of three separate experiments normalized to each mutant's control measured under vehicle treated conditions (100%). Results in A and B reflect mean values±SEM of three separate experiments normalized to each mutant's level under vehicle treated conditions (100%). Data were analyzed by a One-Way ANOVA with post-hoc Bonferonni tests comparing variant to hSERT 8BrcGMP/anisomycin responses with p<0.05 taken as significant.

FIG. 4—Impact of 8BrcGMP on hSERT surface binding. HeLa cells transfected with hSERT or hSERT coding variants were treated with either 100 μM 8BrcGMP+/−H8 or vehicle for 1 hr. Cells were subjected to cell surface [¹²⁵I]RTI-55 (5 nM) binding with 5-HT (100 μM) as displacer. Data were analyzed by a One-Way ANOVA with post-hoc Bonferonni tests comparing variant to hSERT anisomycin responses with p<0.05 taken as significant.

FIG. 5—Altered PKG/p38 MAPK sensitivity of 56Ala is evident in native lymphocytes and may involve altered transporter phosphorylation. (FIG. 5A) Representative total extract immunoblot (upper) and autoradiogram from SERT immunoprecipitations. Lymphocytes were genotyped and cultured as described in Methods and assessed for 5-HT uptake regulation as described for transfected HeLa cells. Data presented derive from individual lymphocyte lines of determined genotype. Findings were replicated in a separate set of genotyped lines with equivalent results. Uptake levels for each genotype with vehicle treated conditions were taken as 100%. Transport activities were analyzed by a Two-Way ANOVA with post-hoc Bonferroni tests with p<0.05 taken as significant. (FIG. 5B) Quantitation of SERT labeling from phosphorylation studies (n=3). hSERT Gly56Ala variant displays altered basal phosphorylation and sensitivity to 8BrcGMP. SERTs expressed in transfected HeLa cells were examined 36 hrs after transfection. Values are expressed as mean±S.E.M. *, p<0.01 versus WT-vehicle, ##, p<0.05 versus WT-vehicle by one-way ANOVA with Bonferroni post-hoc analysis.

FIG. 6—Saturation kinetic analysis of 5-HT transport by hSERT, Pro339Leu, and Ile425Val. Plots are derived from mean activities obtained in 3 or more separate studies. Kinetic values obtained were the following: hSERT, Vₘₐₓ, 416.2+19 fmol/well/min: Kₘ, 1.00+0.17 μM; Pro339Leu, Vₘₐₓ, 1.2+0.11 fmol/well/min: Kₘ, N.D.; Ile425Val, Vₘₐₓ, 789.5+28 fmol/well/min:Kₘ, 0.56+0.13 μM.

FIGS. 7A-B—Impact of SERT coding variants on total and cell surface [¹²⁵I]RTI-55 binding. HeLa cells transiently transfected with hSERT or one of the SERT coding variants were subjected to intact cell binding assays at 4° C. with the cocaine analog [¹²⁵I]RTI-55 (5 nM) as described in Methods. (FIG. 7A) Total binding values as defined with paroxetine (10 μM) as displacer. (FIG. 7B) Surface labeling by [¹²⁵I]RTI-55 as defined with 5- HT (100 μM) as displacer. In vehicle-treated cells, hSERT total binding (fmol/10⁵) was 618.2+27.9, and the surface binding was 173.8+6.6. Results for FIG. 7A and FIG. 7B reflect mean values±SEM of three separate experiments normalized to hSERT(100%). Binding levels were analyzed via a One-Way ANOVA followed by post-hoc Dunnett's tests comparing mutant means to hSERT, with p<0.05(*) taken as significant.

FIG. 8—Modulation of hSERT and coding variants by phorbol ester. Transfected cells treated with 10 or 100 μM β-PMA for 15 min in the presence or absence of the PKC antagonist BIM were assayed for 5-HT transport as described in Methods. P339L was not tested due to low basal transport activity. Results represent mean values±SEM of three separate experiments normalized to each mutant's vehicle treated control(100%). Data were analyzed using a Two-Way ANOVA followed by a post-hoc Bonferonni test to evaluate differences in PMA effects comparing hSERT to each coding variant with p<0.05(*) taken as significant.

FIGS. 9A-D—Effects of p38 MAPK activation on 5-HT uptake in RBL-2H3 cells. (FIG. 9A) Dose response. Cells were treated with varying concentrations of anisomycin for 10 min prior to transport assay. (FIG. 9B) Time course. Influence of pretreatment duration on anisomycin (1 μm) stimulation of 5-HT uptake. Values in A and B are expressed as the mean of at least three experiments±S.E. *, p<0.05; **, p<0.01 versus controls (one-way ANOVA, Dunnett). (FIG. 9C) Effects of various p38 MAPK activators. RBL-2H3 cells were incubated with the specific p38 MAPK inhibitor SB203580 (10 μm) or vehicle for 15 min, followed by the treatment with anisomycin (1.0 μm), hydrogen peroxide (20 μm), or UV radiation (4×10⁴ μJ/cm²). Values are expressed as mean values (n=4)±S.E. **, p<0.01 versus control (one-way ANOVA, Dunnett). (FIG. 9D) Inhibitor sensitivity of uptake stimulation. RBL-2H3 cells were preincubated with vehicle, p38 MAPK inhibitor SB202190 (10 μm) the inactive analog SB202474 (10 μm), or the c-Jun NH₂-terminal kinase inhibitor curcumin (10 μm) for 15 min, followed by the treatment with anisomycin (1.0 μm, 10 min). Values are expressed as the mean of at least three experiments±S.E. **, p<0.01 versus controls (one-way ANOVA, Dunnett).

FIG. 10—Anisomycin triggers p38 MAPK phosphorylation in RBL-2H3 cells in an SB203580-sensitive manner. RBL-2H3 cells were seeded in a 96-well plate and cultured overnight. The cells were then treated with vehicle (assay buffer) or SB203580 (10 μm) for 15-min followed by another 5-min treatment with anisomycin or NECA at the concentration indicated. The cells were fixed, and an in-cell Western assay was conducted as detailed under “Experimental Procedures.” Shown here is the average intensity of fluorescence for labeling activated p38 MAPK. Values are expressed as the mean±S.E. (n=3). *, p<0.05; **, p<0.01 versus vehicle control (one-way ANOVA, Bonferroni). ANS, anisomycin; SB, SB203580.

FIGS. 11A-C—Effect of p38 MAPK siRNA transfection on SERT activity and p38 MAPK protein expression. RBL-2H3 cells were transfected with vehicle, transfection reagent (Trans-It), or siRNA (100 n_{M}). 5-HT uptake and Western blots were performed 48 h following the transfection. (FIG. 11A) siRNA transfection abolished anisomycin-induced 5-HT uptake without affect basal SERT activity (n=4). (FIG. 11 B) Western blot (a representative from three experiments) showing the expression level of p38 MAPK. (FIG. 11C) Quantification of p38 MAPK band density (n=3). Values are expressed as the mean±S.E. **, p<0.01 versus vehicle control (one-way ANOVA, Bonferroni). ANS, anisomycin; SB, SB203580.

FIG. 12—Biogenic amine transporters are not equivalently sensitive to p38 MAPK activation. CHO cells were transfected separately with human or mouse SERT, human NET, or human DAT cDNAs and cultured for 24 h before anisomycin application and uptake assay. The transfected cells were preincubated with SB203580 for 15 min prior to the 10-min treatment with anisomycin. *, p<0.05; **,p<0.01 versus respective control (n=5, one-way ANOVA, Bonferroni). ANS, anisomycin; SB, SB203580.

FIGS. 13A-C—8-Bromo-cGMP and/or anisomycin stimulates 5-HT uptake in serotonergic RN46A cells and human platelets. (FIG. 13A) Cells were preincubated with vehicle or DT-2 (1.0 μm) for 15 min followed by the treatment with 8-bromo-cGMP at the indicated concentrations for 10 min prior to transport assay. (FIG. 13B) Cells were preincubated with vehicle or SB203580 (10 μm) for 10 min, followed by treatment with anisomycin at the indicated concentrations for 10 min prior to the transport assay. Values are expressed as the mean of three experiments±S.E. **, p<0.01 versus control (one-way ANOVA, Dunnett). (FIG. 13C) Anisomycin-induced stimulation of 5-HT uptake in human platelets. Platelets were treated with anisomycin (1 μm) for 10 min in the presence/absence of SB203580 (10 μm), followed by a 5-HT uptake assay. Values are expressed as the mean±S.E. (n=3). *, p<0.05 versus control (Student's t test).

FIGS. 14A-C—Saturation kinetic and competition binding analyses of anisomycin stimulation. (FIG. 14A) RN46A cells were treated with anisomycin (1 μm) or with vehicle for 10 min prior to transport assays. (FIG. 14B) RBL-2H3 cells were treated with anisomycin (1 μm) or with vehicle for 10 min prior to transport assays. (FIG. 14C) RBL-2H3 cells were treated with vehicle, SB203580 (10 μm), or anisomycin (1 μm) for 10 min prior to the RTI-55 binding assay. 5-HT at varying concentrations as indicated in the figure was used to compete with surface binding of RTI-55. Data were fit in both FIG. 14A and FIG. 14B to a Michaelis-Menten equation (single binding site) to derive 5-HT Kₘ and Vₘₐₓ values and fit in FIG. 14C to a single competition site equation to derive Kᵢ. Values are expressed as mean values (n=3)±S.E. *, p<0.05 versus control (Student's t test). ANS, anisomycin; SB, SB203580.

FIGS. 15A-B—Impact of anisomycin stimulation on surface SERT. (FIG. 15A) anisomycin fails to induce an alteration in SERT surface density. RBL-2H3 cells were preincubated with vehicle or anisomycin (ANS; 1 μm) and/or fostriecin (FST; 5 n_{M}) or NECA (1 μm) for 10 min before measurement of 5-HT-sensitive [¹²⁵I]RTI-55 binding. NECA triggered a significant increase in binding, whereas anisomycin and/or fostriecin did not impact the binding. Neither pretreatment influenced total binding. (FIG. 15B) MTSET inactivation of surface SERTs eliminates anisomycin-induced stimulation of SERT. RBL-2H3 cells were preincubated with vehicle or with MTSET (10 mM) for 10 min on ice and then washed once with uptake assay buffer and incubated with vehicle or SB203580 (10 μm, 15 min), followed by treatment with anisomycin (1 μm, 10 min) prior to the 5-HT uptake assay. Values are expressed as percentages of control values (n=3)±S.E. **, p<0.01 (one-way ANOVA, Dunnett).

FIGS. 16A-B—Inhibitors of guanylyl cyclase and PKG fail to block anisomycin-stimulated 5-HT uptake. (FIG. 16A) RBL-2H3 cells were preincubated with vehicle, ODQ (10 μm), H8 (0.1 μm), or DT-2 (1.0 μm) for 10 min, followed by the application of anisomycin (1 μm) or NECA (1 μm, as positive control). Both ODQ and H8/DT-2 abolish NECA-induced 5-HT uptake but do not impact anisomycin-stimulated uptake (n=5). (FIG. 16B) RN46A cells were preincubated with vehicle, ODQ (10 μm), H8 (0.1 μm), DT-2 (1.0 μm), or SB203580 (10 μm) for 10 min, followed by the application of anisomycin (1 μm). Only SB203580 blocks anisomycin-induced 5-HT uptake (n=3). Values are expressed as mean±S.E. *, p<0.05; **, p<0.01 versus vehicle control (one-way ANOVA, Dunnett).

FIGS. 17A-B—Inhibitors of PP2A block anisomycin stimulation of SERT activity. (FIG. 17A) RBL-2H3 cells were preincubated with 20 or 100 n_{M} calyculin A for 15 min followed by the treatment with anisomycin (1 μm, 10 min). (FIG. 17B) Cells were preincubated with fostriecin at the indicated concentrations for 15 min prior to anisomycin treatment. Both calyculin A and fostriecin block the anisomycin-induced increase of 5-HT uptake. At higher concentrations, these agents also inhibit basal 5-HT uptake. Values are expressed as mean values (n=3)±S.E. *, p<0.05; **, p<0.01 versus control (one-way ANOVA, Dunnett).

### DESCRIPTION OF ILLUSTRATIVE EMBODIMENTS

SERT proteins can be rapidly regulated by multiple G-protein coupled receptors and protein kinase-linked pathways, including those triggered by activation of protein kinase C (PKC), protein kinase G (PKG) and possibly p38 mitogen activated protein kinase (MAPK) (Ramamoorthy et al., 1998; Ramamoorthy and Blakely, 1999; Zhu et al., 2004a,b; Samuvel et al., 2005; Zhu et al., 2005). Phosphorylation and downregulation of SERT through the PKC-linked pathway is sensitive to extracellular 5-HT (Ramamoorthy and Blakely, 1999), revealing an intrinsic capacity for temporal integration of ongoing 5-HT clearance demand with modulatory inputs. The importance of SERT in presynaptic 5-HT homeostasis, synaptic 5-HT clearance and psychoactive drug action has raised questions as to whether the hSERT gene exhibits functional polymorphisms that impact expression and activity in vivo (Murphy et al., 2004).

A number of disorders including anxiety, major depression, OCD, autism, and IBS have been associated with a common, functional variant in the hSERT promoter termed the 5HTTLPR (Murphy et al., 2004; Hahn and Blakely, 2002; Caspi et al., 2003). Less attention has been given to the functional status of hSERT coding variants. In an early report, Lesch and coworkers identified a single Leu255Met allele (Di Bella et al., 1996). Glatt and colleagues (Glatt et al., 2001) greatly expanded the list of known variants in a study of 450 nonclinical subjects, revealing 9 new coding variants, but only one, Gly56Ala, was found more than once and still at a frequency of less than 0.5% (4/900 chromosomes; Table 1). Gly56Ala was also identified at low frequency by Cargill et al. (1999), along with Lys605Asn, also found by Glatt et al. (2001). None of these initial studies characterized the function of hSERT coding variants. Possibly, they could impact hSERT function and provide clues to pathways contributing risk for 5-HT-linked clinical phenotypes. By analogy, the inventors identified Ala457Pro in hNET in a single family with Orthostatic Intolerance (01) (Shannon et al., 2000). Despite the rarity of this variant, its segregation with tachycardia and plasma catecholamines provides important evidence that idiopathic OI likely involves a hypernoradrenergic state.

TABLE 1
Human SERT Nonsynonymous Variants

| Nucleotide | Nucleotide | | | | |
|---|---|---|---|---|---|
| Change in | Change in | Location | | | |
| NCBI Sequence | Protein | in SERT | Position | Allele | Amino Acid |
| Deposit | Sequence | Protein | in Exon | Frequency | Change |
| A483G | A10G | N term | Exon 2 | 1/900^{c} | Thr4Ala^{c} |
| G640C | G167C | N term | Exon 2 | 5/114^{b}; 4/900^{c} | Gly56Ala^{b,c} |
| G1074A | G643A | ECL2 | Exon 4 | 1/900^{c} | Glu215Lys^{c} |
| C1999A | C763A | TMD4 | Exon 5 | 1/200(Dep)^{a} | Leu255Met^{a} |
| C262T | C878T | TMD5 | Exon 6 | 1/900^{c} | Ser293Phe^{c} |
| C767T | C1016Y | TMD6 | Exon 7 | 1/900^{c} | Pro339Leu^{c} |
| C258A | C1084A | TMD7 | Exon 8 | 1/900^{c} | Leu362Met^{c} |
| A318G | A1273G | TMD8 | Exon 9 | 1/900^{c}, 2/60(OCD)^{d} | Ile425Val^{c,d} |
| A469C | A1815C | C term | Exon 13 | 1/114^{b}; 1/900^{c} | Lys605Asn^{b,c} |
| C274T | C1961T | C term | Exon 14 | 1/900^{c} | Pro621Ser^{c} |

^{a}Di Bella et al. (1996)
^{b}Cargill et al. (1999)
^{c}Glatt et al. (2001)
^{d}Ozaki et al. (2003)

### I. THE PRESENT INVENTION

The increasing awareness that rare, functional alleles can define disrupted pathways bearing other disease susceptibility genes (Pritchard, 2001; Cohen et al., 2004) encouraged the inventors to perform a comprehensive, functional evaluation of known hSERT coding variants. Each of the major SERT alleles studied is highly conserved across currently sequenced mammalian SERTs (Table 2). Conservation has been demonstrated as one predictor of functional perturbations (Shu et al., 2003). Overall, the inventors found that 7 of the 10 variants bore functional perturbations including altered protein expression and basal 5-HT uptake, cocaine and antidepressant recognition, or loss of regulation. Among the changes observed is a striking pattern of regulatory disruption, wherein half of the hSERT variants, including all four that are present on cytoplasmic domains, appear specifically refractory to PKG and p38 MAPK-linked signaling pathways.

TABLE 2
Sequence Conservation at Sites of Human SERT Coding Variants

| | Thr4 | Gly56 | Glu215 | Leu255 | Ser293 | Pro339 | Leu362 | Ile425 | Lys605 | Pro621 |
|---|---|---|---|---|---|---|---|---|---|---|
| hSERT | T | G | E | L | S | P | L | I | K | P |
| rSERT | T | G | Q | L | S | P | L | I | K | P |
| mSERT | T | G | Q | L | S | P | L | I | K | P |
| gpSERT | T | G | E | L | S | P | L | I | K | P |
| bovSERT | T | G | E | L | S | P | L | I | K | P |
| dSERT | S | T | E | L | I | P | L | I | R | V |
| ceSERT | W | H | D | M | S | P | V | F | Y | S |
| hNET | A | L | L | L | F | A | L | V | W | I |
| hDAT | S | T | G | L | T | V | I | V | R | V |
| hGAT1 | S | T | T | V | R | V | A | L | Y | I |

Abbreviations: hSERT, human serotonin transporter (SERT); rSERT, rat; mSERT, mouse, gpSERT, guinea pig; boxSERT, bovine; dSERT, Drosophila melanogaster; ceSERT, C. elegans; hNET, human norepinephrine transporter; hDAT, human dopamine transporter; hGAT1, human GABA transporter type 1.

The inventors found two variants whose changes in 5-HT uptake capacity were accompanied by parallel changes in total and/or cell surface protein expression. Pro339Leu exhibits a major loss of mature, N-glycosylated protein consistent with improper folding leading to inefficient biosynthetic progression or rerouting to degradative pathways. Relative to the amount that reaches the surface, a greater functional loss is observed, suggesting further disruption of the 5-HT translocation mechanism. Pro339Leu lies in TM6, a domain suggested to participate in transporter oligomerization (Hastrup et al., 2001). Whereas Pro339 is conserved down to C. elegans, the residue is not conserved in NET and DAT, consistent with a more specific role in 5-HT translocation or unique aspects of the transporter's biosynthesis not tested in our studies.

Ile425Val presented the opposite phenotype, with greatly enhanced transport activity coupled to increased cell surface density. Recently, Ozaki and coworkers (Ozaki et al., 2003) identified this variant in two families with a complex psychiatric phenotype including OCD and Asperger's Syndrome. Of the nine affected subjects in these two families, seven carried a single copy of the allele in the background of a homozygous 5HTTLPR L/L genotype. The inventors were unable to reproduce the loss of regulatory sensitivity for Ile425Val reported by Kilic and coworkers (Kilic et al., 2003), though they did observe the reported increase in basal 5-HT transport capacity. The inventors also found, using hSERT inducible cell lines (unpublished findings), that regulation by the PKG/p38 MAPK pathways become less evident or is non-detectible with higher level expression of hSERT, and thus a variant such as Ile425Val, bearing constitutively elevated surface density and 5-HT uptake, may more readily saturate the regulatory machinery upon heterologous expression. Regardless, both studies agree that Ile425Val represents a hypermorphic mutation whose altered activity, presuming similar effects in vivo, may constitutively inappropriately elevate synaptic 5-HT clearance.

The most striking finding in the current report is the complete lack of sensitivity of five of the ten hSERT variants to acute activators of PKG or p38 MAPK. This loss of sensitivity does not correlate with changes in basal 5-HT transport activity, as both Pro339Leu and Ile425Val, which show hypomorphic and hypermorphic phenotypes, respectively, demonstrated stimulation by 8BrcGMP and anisomycin. Additionally, all variants displayed a relatively robust sensitivity to phorbol ester-triggered downregulation.

Finally, though there were changes noted for cocaine and antidepressant recognition, these changes were not highly correlated with loss of transporter stimulation. In fact, the greatest number of changes in antagonist recognition occurred within TMs 4-8 whereas 4 of the 5 PKG/p3 8 MAPK insensitive variants lie in either the cytoplasmic NH2 or COOH termini. At these latter sites, it is reasonable to speculate that variants may disrupt interactions with accessory proteins, which in past years has grown to include syntaxin 1A (Haase et al., 2001), PP2Ac (Bauman et al., 2000), Hic-5 (Carneiro et al., 2002), and MacMARCKS (Jess et al., 2002).

The loss of regulation by Glu215Lys is more difficult to explain, but could suggest a conformational linkage of EL2 to regions of TM3 linked to 5-HT recognition (Chen et al., 1997). EL2 movement, when limited by zinc coordination, blocks substrate transport in homologous DAT proteins (Norregaard et al., 1998), and thus sequence variation in this loop may perturb regulatory conformational changes propagated from intracellular domains to the substrate binding site. In this regard, the p38 MAPK pathway triggers an increase in 5-HT affinity, as assessed in antagonist binding assays (Zhu et al., 2005).

Measurement of Gly56 and Ala56 hSERT lymphocyte mRNA by real-time PCR revealed no differences in SERT mRNA levels (data not shown) consistent with a post-transcriptional origin for the deficts in transporter regulation observed. hSERT proteins exhibit basal phosphorylation and become further phosphorylated in response to activators of PKA, PKC and PKG (Ramamoorthy et al., 1998). To consider the integrity of hSERT PKG phosphorylation, the inventors explored the extent of phosphorylation of the Gly56Ala variant in transiently transfected HeLa cells and obtained evidence that the variant exhibits elevated basal phosphorylation and cannot be further phosphorylated in response to 8BrcGMP treatments. The Gly56Ala variant, possibly through disrupted phosphatase interactions (Bauman et al., 2000), may lack normal inhibitory mechanisms restricting basal phosphorylation. Alternatively, the Gly56Ala variant may impart a gain-of-function phenotype that leads to elevated basal phosphorylation. For example, 5-HT gated channel activity is unmasked in SERT proteins by elimination of regulatory syntaxin 1A interactions (Di Bella et al., 1996) and possibly, changes in basal phosphorylation are indicative of novel states that directly or indirectly enhance basal phosphorylation of SERT. Additional studies are needed to expand this effort to p38 MAPK stimulation, clarify which of the several kinases targeting SERT supports enhanced basal phosphorylation, and to extend phosphorylation studies to the other affected variants.

Loss of regulation through the PKG pathway may leave the endocytic mechanisms supported by PKC-linked pathways (Ramamoorthy et al., 1998; Qian et al., 1997) unopposed. Four of the five PKG/p38 MAPK non-responsive alleles (Thr4Ala, Gly56Ala, Lys605Asn, and Pro621Ser) actually demonstrated significantly enhanced phorbol ester-mediated down-regulation, further enhancing this possibility. Enhanced sensitivity to phorbol ester-mediated down-regulation may also be a clue as to why the variants that fail to show enhanced 5-HT uptake after 8BrcGMP treatments actually show decreased [¹²⁵ I]RTI-55 surface binding. Signals that trigger the PKG-dependent shuttling of new transporters to the surface may also enhance endocytic recycling rates (Melikian, 2004) possibly through crosstalk with PKC-linked pathways. As such, diminished [¹²⁵I]RTI-55 binding to the variants may report a stabilized, partial conformational transition on the endocytic limb. The inventors have recently reported that platelet SERT exhibits surface-resident inactive states (Jayanthi et al., 2005) and it is conceivable that [¹²⁵I]RTI-55 binding might report a state occupied prior to uptake inactivation. Although these ideas remain speculative at best, the 5 variants lacking uptake stimulation by PKG/p38 MAPK activators would appear to be useful in probing different steps in the complex regulatory pathways that ultimately establishes 5-HT uptake capacity.

Since the changes reported here are genetically-encoded and SERT expression occurs early in development, the non-responsive alleles could compromise the ability of SERT to modulate in response to environmental demands and elevate risk for developmental disorders linked to altered 5-HT signaling (Lebrand et al., 1998; Persico et al., 2001; Ansorge et al., 2004). Modulation of SERT activity in neonatal animals has lasting effects on emotional behavior in adults and genetic variation at the hSERT promoter has been reported to interact with early childhood stressors to influence risk for depression and suicide in later life (Caspi et al., 2003). Possibly, carriers of regulatory nonresponsive hSERT alleles may be at greater risk for adult onset disorders arising from inappropriate hSERT activity at critical periods in development. The Gly56Ala allele though uncommon, is still carried by approximately 1:200 Caucasian subjects (Glatt et al., 2001), representing more than a million Americans. Interestingly, disrupted 5-HT signaling has long been discussed as a potential underlying determinant of altered development and behavior in autism (Cook and Leventhal, 1996; Ciaranello, 1982; Piven et al., 199 1).

Because of prior studies noting linkage of autism to the SERT locus at 17q11.2 (McCauley et al, 2004) and the inventors' access to a large collection of autism family DNA samples with matching lymphocyte lines, they genotyped subjects for the Gly56Ala allele and accessed banked cell lymphocyte lines to determine the functional impact of the variant allele within native hSERT expressing cells. Although details of allelic segregation with the autism phenotype must be confirmed through dedicated studies, the inventors found the Gly56Ala allele at a frequency of 2.3% in 120 families selected on the basis of linkage to autism at 17q11.2, a >5-fold increase in allele frequency over the Gly56Ala frequency published by Glatt and colleagues in a study of 450 nonclinical subjects (Glatt et al., 2001). The homozygous Gly56Ala lines the inventors identified derive from two male subjects with autism. Recent evidence for linkage of markers at 17q11.2 to autism (McCauley et al., 2004; Yonan et al., 2003; IMGSAC, 2001) and male-specific autism risk in particular (Stone et al., 2004), argue for further evaluation of the phenotype of hSERT Gly56Ala carriers, as well as a directed search for additional hSERT alleles that can similarly impact transporter regulation.

### II. BIOGENIC AMINE TRANSPORTERS (BAT)

The present invention exemplifies the modulation of SERT with respect to the p38 mitogen-activated protein kinase (MAPK) pathway. Given the significant similarities between SERT and the norepinephrine transporter (NET) in terms of p38 MAPK signalling, one may logically extend these studies to the NET pathway as well.

A. Serotonin Transporter (SERT)

The serotoninergic system modulates numerous behavioral and physiological functions and has been associated with control of mood, emotion, sleep and appetite. Synaptic serotonin (SE), also called 5-hydroxytryptamine or 5HT, concentration is controlled by the serotonin transporter (SERT) which is involved in reuptake of serotonin into the pre-synaptic terminal. In several studies, 5HT uptake and/or transport sites have been found to be reduced in platelets of patients suffering from depression and reduced in post-mortem brain samples of depressed patients and suicide victims (Meltzer et al., 1981; Suranyi-Cadotte et al., 1985; Briley and Moret, 1993; Paul et al., 1981; Perry et al., 1983). The cloning of the human SERT protein by Ramamoorthy et al. (1993), shows that human SERT is encoded by a single gene that is localized to chromosome 17q 11.1-17q12 and encodes for a 630-amino acid protein. The hSERT is a Na⁺- and Cl⁻-coupled serotonin transporter and has been found to be expressed on human neuronal, platelet, placental, and pulmonary membranes (Ramamoorthy et al., 1993).

B. Norepinephrine Transporter

The norepinephrine transporter (NET) is an antidepressant-sensitive transporter located on plasma membranes of noradrenergic neurons and other specialized cells that removes norepinephrine (NE) from the synapse to terminate the actions of NE. It contains of 617 amino acids and has 12 transmembrane domains. This conformation is similar to that of other membrane-associated proteins that are responsible for ion and solute transport.

NET removes not only NE but, in regions such as the rat prefrontal cortex, a major portion of surplus dopamine as well. Peripherally, NET is perhaps most important in the heart. In humans, approximately 92% of cardiac NE removal occurs via NET, with the remainder eliminated by extraneuronal carriers and circulatory dissipation. Pharmacological blockade of NET increases extraneuronal concentrations of its substrate(s), a mechanism employed in the treatment of depression and anxiety, although autonomic side effects are common. Impaired NET function is linked to familial orthostatic intolerance, an autonomic disorder characterized by excessive tachycardia, minimal blood pressure changes, and increased NE on assumption of upright posture.

C. Protein Compositions

The term “protein” or “amino acid” composition encompasses amino acid sequences comprising at least one of the 20 common amino acids in naturally synthesized proteins, or at least one modified or unusual amino acid. It is also well understood that where certain residues are shown to be particularly important to the biological or structural properties of a protein, polypeptide or peptide, e.g., residues in binding regions or active sites, such residues may not generally be exchanged. In this manner, functional equivalents are defined herein as those peptides which maintain a substantial amount of their native biological activity.

The present invention provides a variety of SERT molecules, including those that are “altered” when compared to wild-type sequences. Such variants include those that have the changes listed in Table 1. For comparison purposes, the wild-type sequence for human SERT is set forth in SEQ ID NO:1, and that for mouse SERT is set forth in SEQ ID NO:3. Such variants may also be insertional or deletion variants, and the methods of preparing these variants are well known in the art. Insertional variants can also include fusion proteins, or hybrid proteins containing sequences from other proteins and polypeptides which are homologues of the polypeptide.

Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Non-conservative changes are more likely to have any impact on the function of the molecules.

### III. NUCLEIC ACID MOLECULES

A. Nucleic Acids Encoding Serotonin Transporter

The present invention also provides SERT-encoding nucleic acids. Nucleic acids of the present invention may be derived from genomic DNA, complementary DNA (cDNA). More particularly, the present invention provides synthetic nucleic acid sequences comprising the amino acid sequences of the human serotonin transporter. Nucleic acids of the present invention also concern isolated DNA segments encoding wild-type, polymorphic or mutant serotonin transporter proteins, polypeptides or peptides. Mutants are designated in comparison to the wild-type human SERT sequence, SEQ ID NO:2.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 20, of about 50 to about 90, of about 100 to about 200, of about 210 to about 300, of about 310 to about 350, of about 360, to about 400, of about 410 to about 450, of about 460 to about 500, of about 510 to about 550, of about 560 to about 600, of about 610 to about 650, of about 660 to about 700, of about 710 to about 750, of about 760 to about 800, of about 810 to about 850, of about 860 to about 900, about 1000, about 1100, about 1200, about 1300, about 1400, about 1500, about 1600, about 1700, about 1800, about 1900 or greater nucleotide residues in length. Those of skill will recognize that in cases where the nucleic acid region encodes a serotonin transporter peptide, polypeptide or protein, the nucleic acid region can be quite long, depending upon the number of amino acids in the serotonin transporter molecule.

It is contemplated that the serotonin transporter may be encoded by any nucleic acid sequence that encodes the appropriate amino acid sequence. The design and production of nucleic acids encoding a desired amino acid sequence is well known to those of skill in the art, using standardized codon tables (Table 3). In preferred embodiments, the codons selected for encoding each amino acid may be modified to optimize expression of the nucleic acid in the host cell of interest. The term “functionally equivalent codon” is used herein to refer to codons that encode the same amino acid, such as the six codons for arginine or serine, and also refers to codons that encode biologically equivalent amino acids. Codon preferences for various species of host cell are well known in the art. Codons preferred for use in humans, are well known to those of skill in the art (Wada et al., 1990). Codon preferences for other organisms also are well known to those of skill in the art (Wada et al., 1990, included herein in its entirety by reference) and can be found on the internet at the Codon Usage Database website.

TABLE 3

| Amino Acid | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In certain embodiments, the invention concerns isolated DNA segments and recombinant vectors that include within their sequence a contiguous nucleic acid sequence encoding the amino acid sequences shown in SEQ ID NO: 2. As used herein, the term “DNA segment” refers to a DNA molecule that has been isolated free of total genomic DNA of a particular species. Therefore, a DNA segment encoding serotonin transporter refers to a DNA segment that contains wild-type, polymorphic or mutant serotonin transporter coding sequences yet is isolated away from, or purified free from, total mammalian genomic DNA. Included within the term “DNA segment,” are DNA segments and smaller fragments of such segments, and also recombinant vectors, including, for example, plasmids, cosmids, phage, viruses, and the like.

Similarly, a DNA segment comprising an isolated or purified serotonin transporter gene refers to a DNA segment including serotonin transporter protein, polypeptide or peptide coding sequences and, in certain aspects, regulatory sequences, isolated substantially away from other naturally occurring genes or protein encoding sequences. In this respect, the term “gene” is used for simplicity to refer to a functional protein, polypeptide or peptide encoding unit. As will be understood by those in the art, this functional term includes both genomic sequences, cDNA sequences and engineered segments that express, or may be adapted to express, proteins, polypeptides, domains, peptides, fusion proteins and mutants of serotonin transporter encoded sequences.

“Isolated substantially away from other coding sequences” means that the gene of interest, in this case the serotonin transporter gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

In particular embodiments, the invention concerns isolated DNA segments that encode a serotonin transporter protein, polypeptide or peptide that includes within its amino acid sequence a contiguous amino acid sequence in accordance with, or essentially as set forth in, SEQ ID NO: 2.

The term “a sequence essentially as set forth in SEQ ID NO: 2” means that the sequence substantially corresponds to a portion of SEQ ID NO: 2 and has relatively few bases that are not identical to, or a biologically functional equivalent of (i.e., encode amino acid), SEQ ID NO: 2.

It will also be understood that nucleic acid sequences may include additional residues, such as additional 5′ or 3′ sequences, and yet still be essentially as set forth in one of the sequences disclosed herein. The addition of terminal sequences particularly applies to various non-coding sequences flanking either of the 5′ or 3′ portions of the coding region or may include various internal sequences, i.e., introns, which are known to occur within genes.

Excepting intronic or flanking regions, and allowing for the degeneracy of the genetic code, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of nucleotides that are identical to the nucleotides of SEQ ID NO: 2.

If desired, one also may prepare fusion proteins and peptides, e.g., where the nucleic acid encoding a serotonin transporter are aligned within the same expression unit with other proteins or peptides having desired functions, such as for purification or immunodetection purposes (e.g., proteins that may be purified by affinity chromatography and enzyme label coding regions, respectively).

In addition to the “standard” DNA and RNA nucleotide bases, modified bases are also contemplated for use in particular applications of the present invention. A table of exemplary, but not limiting, modified bases is provided herein (Table 4).

TABLE 4
Modified and Unusual Amino Acids

| Abbr. | Amino Acid |
|---|---|
| Aad | 2-Aminoadipic acid |
| Baad | 3-Aminoadipic acid |
| Bala | -alanine, -Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In addition to nucleic acids encoding the serotonin transporter, the present invention encompasses complementary nucleic acids that hybridize under high stringency conditions with such coding nucleic acid sequences. High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

B. Vectors

It is contemplated in the present invention, that virtually any type of vector may be employed in any known or later discovered method to deliver nucleic acids encoding a serotonin transporter. Such vectors may be viral or non-viral vectors as described herein, and as known to those skilled in the art. U.S. Pat. Nos. 5,312,734, 5,418,162, and 5,424,185, all incorporated herein by reference, describe nucleic acids, vectors, and host cells used to express various neurotransmitter transporters in cells.

1. Expression Constructs

A vector in the context of the present invention refers to a carrier nucleic acid molecule into which a nucleic acid sequence encoding a serotonin transporter can be inserted for introduction into a cell and thereby replicated. A nucleic acid sequence can be exogenous, in that it is foreign to the cell into which the vector is being introduced; or that the sequence is homologous to a sequence in the cell but positioned within the host cell nucleic acid in which the sequence is ordinarily not found. One of skill in the art would be well equipped to construct a vector through standard recombinant techniques as described in Sambrook et al. (2001); Maniatis et al. (1990); and Ausubel et al. (1994) (each incorporated herein by reference).

It is contemplated in the present invention, that virtually any type of vector may be employed in any known or later discovered method to deliver nucleic acids encoding a serotonin transporter peptide, polypeptide or protein, or constructs thereof. Such vectors may be viral or non-viral vectors as described herein, and as known to those skilled in the art.

An expression vector of the present invention refers to any type of genetic construct comprising a nucleic acid coding for a RNA capable of being transcribed. In some cases, RNA molecules are translated into a protein, polypeptide, or peptide. An expression construct comprising a nucleic acid encoding a serotonin transporter peptide, polypeptide, or protein may comprise a virus or engineered construct derived from a viral genome and may also comprise a natural intron or an intron derived from another gene. In other cases, these sequences are not translated as in the case of antisense molecules or ribozymes production. Expression vectors can contain a variety of control sequences, which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well, and are described herein. Additionally, as set forth above one may also use mutant versions, isoforms, and other variants of any neurotransmitter transporter in the methods of the invention. The foregoing section provides a general description of how exogenous expression may be achieved.

Expression requires that appropriate signals be provided in the vectors, which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

a. Promoters and Enhancers

Expression requires that appropriate signals be provided in the vectors, and which include various regulatory elements, such as enhancers/promoters from both viral and mammalian sources that drive expression of the genes of interest in host cells.

Throughout this application, the term “expression construct” is meant to include any type of genetic construct containing a nucleic acid coding for a gene product in which part or all of the nucleic acid encoding sequence is capable of being transcribed and translated into a polypeptide product. An “expression cassette” is defined as a nucleic acid encoding a gene product under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

By attaching a tissue-specific or cell-specific promoter region of a nucleic acid to a reporter or a detectable marker, one can obtain tissue-specific or cell-specific expression. The present invention particularly contemplates the use of the serotonin promoter to drive expression of the nucleic acid of interest.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 3 and 4 list several regulatory elements that may be employed, in the context of the present invention, to regulate the expression of the gene of interest.

Additionally, any promoter/enhancer combination (as per the Eukaryotic Promoter Data Base EPDB) may be used to drive expression of the gene. Eukaryotic cells can support cytoplasmic transcription from certain bacterial promoters if the appropriate bacterial polymerase is provided, either as part of the delivery complex or as an additional genetic expression construct.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

The use of internal ribosome entry sites (IRES) elements may be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

C. Polyadenylation and Termination Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells.

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals.

Also contemplated as an element of the expression cassette is a transcriptional termination site. The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

These elements can serve to enhance message levels and/or to minimize read through from the cassette into other sequences.

d. Splicing Sites and Origins of Replication

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997), incorporated herein by reference).

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

e. Multiple Cloning Sites

Vectors can include a multiple cloning site (MCS), which is a nucleic acid region that contains multiple restriction enzyme sites, any of which can be used in conjunction with standard recombinant technology to digest the vector (see Carbonelli et al., 1999; Levenson et al., 1998; and Cocea, 1997; incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

2. Selectable Markers

In certain embodiments of the invention, the cells contain nucleic acid constructs encoding a neurotransmitter transporter may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (see the atcc website on the internet). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5, JM109, and KC8, as well as a number of commercially available bacterial hosts such as SURE® Competent Cells and SOLOPACK Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

D. Viral Transfer

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubinstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubinstein, 1988; Temin, 1986).

The retroviruses are a group of single-stranded RNA viruses characterized by an ability to convert their RNA to double-stranded DNA in infected cells; they can also be used as vectors. Adenoviruses are also typically used as vectors due to their mid-sized genome, ease of manipulation, high titer, wide target cell range and high infectivity. The use of retroviral and adenoviral vectors in eukaryotic gene expression and gene therapy are well known in the art.

Other viral vectors may also be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. These vectors offer several attractive features for various mammalian cells (Friedmann, 1989; Ridgeway, 1988; Baichwal and Sugden, 1986; Coupar et al., 1988; Horwich et al., 1990).

E. Non-Viral Transfer of Nucleic Acids Encoding SERT

There are a number of suitable methods by which nucleic acids encoding amino acid sequences of the serotonin transporter may be introduced or delivered to cells. Virtually any method by which nucleic acids (e.g., DNA, including viral and nonviral vectors) can be introduced into a cell, or an organism may be employed with the current invention, as described herein or as would be known to one of ordinary skill in the art. Several methods for the transfer of expression constructs into mammalian cells include, but are not limited to: direct delivery of DNA by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods.

### IV. SCREENING FOR ALTERATIONS IN SEROTONIN TRANSPORTER FUNCTION

Defects in serotonin transporter are associated with various nervous system disorders including depression, stress disorders, attention deficit disorder, anxiety, obesity, several sleep related disorders and certain neurodegenerative diseases (Edwards, 1993). For example, the biogenic amine transporter which is responsible for inactivation of serotonin is a major target for multiple psychoactive substances including cocaine, amphetamines, methylphenidate (Ritalin™), tricyclic antidepressants and the SSRIs such as fluoxetine (Prozac™). Similarly, drugs that act through SERT can be impacted by alterations in the SERT gene. Thus, it is important to identify which drugs are affected by which SERT polymorphisms.

The present invention therefore provides methods for screening SERT variants to determine their ability to be modulated by various drugs. The drugs may be known therapeutics, or may be members of large libraries of candidate substances. The functions examined include binding, uptake, accumulation, or clearance of the neurotransmitter, its analog or derivative or for some biological aspect of neurotransmitter release, uptake or clearance. Micro-dialysis and amperometry may be used to assay transporter function in vivo (Giros et al., 1996; Galli et al., 1998).

Assays may be conducted in cell free systems such as cellular extracts, cell membrane preparations which may be prepared by lysing cells, in isolated cells, in cells that express endogenous serotonin transporter, in cells that are genetically engineered to express the serotonin transporter, in cells that exogenously or endogenously express mutant or functionally deficient transporters, or in organisms including transgenic animals or animal models of diseases wherein the disease is associated with neurotransmitter transporters.

It will, of course, be understood that all the screening methods of the present invention are useful in themselves notwithstanding the fact that effective candidates may not be found. The invention provides methods for screening for such candidates, not solely methods of finding them.

A. In Vitro Assays

In particular embodiments, the present invention provides a method for screening SERT variants in an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay in this invention is the use of cellular extracts that comprise a neurotransmitter. These may be cell membrane preparations that comprise a neurotransmitter transporter, particularly a serotonin transporter. Another example is a cell-binding assay. While not directly addressing function, the ability of an inhibitor or blocker to bind to a target molecule (in this case the serotonin transporter) in a specific fashion is strong evidence of a related biological effect. For example, binding of a molecule to a serotonin transporter may, in and of itself, be inhibitory, due to steric, allosteric or charge-charge interactions. The serotonin transporter protein may be either free in solution, fixed to a support, expressed in or on the surface of a cell. Either the serotonin transporter or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

B. In Cyto Assays

Various cells and cell lines can also be utilized for screening assays for drug effects on variant serotonin transporters. This includes cells specifically engineered to expresses a SERT variant. Such cells and nucleic acid vectors are described in several sections infra as well as U.S. Pat. Nos. 5,312,734, 5,418,162, and 5,424,185, the contents of which are all incorporated herein by reference. Cells contemplated in the present invention include, but are not limited to, neuronal cells. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

1. Measurement of Transport

In some embodiments, the present invention provides a novel and rapid method for analysis of transport by a serotonin transporter that comprises the measurement of uptake and/or accumulation of serotonin and analogues thereof that are specifically taken up by the transporter. Typically, this is accomplished by measuring the uptake or binding of radiolabeled serotonin (e.g. [³H]serotonin) or a radiolabeled antagonist such as [³H]citalopram, [³H]paroxetine, or [¹²⁵I]RTI-55. Conventional assays involves the uptake of radiolabeled 5HT where antagonist sensitivity is measured for inhibition of serotonin accumulation or the inhibition of labeled antagonist binding to intact cells expressing SERT or to membranes from intact cells expressing SERT. Basically, cells transfected with a SERT construct are washed in assay buffer followed by a preincubation in 37° C. assay buffer containing 1.8 g/L glucose. This is followed by an incubation period, about 10 minutes, at 37° C. in the presence of [³H]-5-HT, or a radiolabeled antagonist such as [³H]citalopram, [³H]paroxetine, or [^{125I}]RTI-55.

a. Scintillation Proximity Assays

Measurement of transport may also be involve scintillation proximity assays, which is used to count the accumulated radiolabel on plates having scintillant embedded in them. Basically, cells are plated at 50% confluence on 0.4-μm pore size 6.5-mm Transwell cell culture filter inserts and grown for 7 days. A cell monolayer growing on the porous membrane of the cell culture filter insert effectively separates each well in the cell culture plate into two chambers. The apical membranes of epithelial cells plated on these filters faces the chamber above the cells and the basolateral membranes face the lower chamber through the filter. After one wash each of the apical (upper chamber) and basolateral (lower chamber) sides of the monolayer with PBS/Ca/Mg, the cells are incubated in PBS/Ca/Mg containing ³H-labeled substrate either in the upper or the lower chamber at 22° C. At the end of the incubation cells are washed either three times from the apical side and once from the basolateral side (when ³H-labeled substrate was present in the upper chamber) or once from the apical side and three times from the basolateral side (when substrate was present in the lower chamber). The apical side of the cells are washed by adding 0.2 ml of ice-cold PBS to the upper chamber and aspirating. The basolateral side of the cells are washed by pipetting ice-cold PBS over the bottoms of the filter inserts. After the washes, the filters with cells attached are excised from the insert cups, submerged in 3 ml of Optifluor scintillation fluid (Packard Instrument Co., Downers Grove, Ill.), and counted in a Beckman LS-3801 liquid scintillation counter. Transport assays on 48-well plates were described previously (Gu et al., 1994).

b. Voltage and Patch Clamp

The present invention also employs a means of determining the serotonin transporter activity or function by measuring the change in movement across a membrane, when the transporter is active. This may be accomplished using the voltage clamp technique, as is well known in the art, this allows the gating properties of the voltage-gated channels to be analyzed.

In short, the voltage clamp technique is a procedure whereby the transmembrane voltage of a membrane segment is rapidly set and maintained at a desired level. Once the membrane potential is controlled, the current flowing through the channels in that segment can be measured.

The patch clamp technique allows the voltage clamp technique to be applied to a small patch of membrane containing a single voltage-sensitive channel. The basic idea behind a patch clamp experiment is to isolate a patch of membrane so small that it contains a single voltage-gated channel. Once this patch of membrane is isolated, the single channel can be voltage clamped. Using this technique, the gating properties of the serotonin transporter can be characterized.

2. Other Methods of Measurement of Transport

Other methods of measurement contemplated in the present invention may involve fluorescence microscopy. This may involve the use of fluorescent substrates, some of which are contemplated to be analogs of other native neurotransmitters.

a. Microscopy

Fluorescent microscopy is used to measure transport using serotonin or analogues thereof which are fluorescent substrates for the serotonin transporter. Cells that either endogenously or exogenously express a serotonin transporter are isolated and plated on glass bottom Petri-dishes or multi-well plates that may typically be coated with poly-L-lysine or any other cell adhesive agent. Cells are typically cultured for three or more days. The culture medium is then aspirated and the cells are mounted on a Zeiss 410 confocal microscope. During the confocal measurement cells remain without buffer for approximately thirty seconds. Background autofluorescence is established by collecting images for ten seconds prior to the addition of the buffer and serotonin or analogues thereof. As serotonin or an analogue thereof has a large Stoke shift between excitation (Iₘₐₓ=488 nm) and emission maxima (Iₘₐₓ=610 nm), the argon laser is tuned to 488 nm and the emitted light filtered with a 580-630 nm band pass filter (Iₘₐₓ=610 nm). The substantial red shift can be exploited to reduce background auto-fluorescence produced in the absence of substrate. The gain (contrast) and offset (brightness) for the photomultiplier tube (PMT) may be set to avoid detector saturation at the higher serotonin concentrations that may be used in certain experiments. The effects of photo-bleaching on serotonin accumulation may also be determined by examining the rate of serotonin accumulation and decay at various acquisition rates. In a constant pool of serotonin, rates as high as 20 Hz (50 msec/image) can be set.

b. Fluorescence Anisotropy Measurements

To evaluate serotonin or analogues thereof binding to the surface membranes, cells expressing a serotonin transporter may be exposed to serotonin or analogues thereof with horizontal polarizer, with the polarizer rapidly switching to the vertical position. Cells may be imaged with alternating polarizations for 3 minutes to measure light intensity in the horizontal (Iₕ) and vertical (Iᵥ) positions in order to calculate the anisotropy ratio, r=(Iᵥ−gIₕ)/(Iᵥ+2gIₕ). The factor g may be determined by using a half wave plate as described by Blackman et al. (1996). In this formulation, r=0.4 implies an immobile light source. Surface anisotropy can be measured at the cell circumference over 1 pixel width (0.625 mm). Cytosolic anisotropy can be measured near the center of the cell, approximately 5 pixel widths from the membrane.

C. Image Analysis

The fluorescent images may be processed using suitable software. For example, fluorescent images may be processed using MetaMorph imaging software (Universal Imaging Corporation, Downington Pa.). Fluorescent accumulation may be established by measuring the average pixel intensity of time resolved fluorescent images within a specified region identified by the DIC image. Average pixel intensity is used to normalize among cells.

d. Single Cell Fluorescence Microscopy

In some embodiments, the invention provides measurement of transporter characteristics at the single-cell level. Single-cell fluorescence microscopy provides a powerful assay to study rapid serotonin uptake kinetics from single cells.

e. Automation

The inventors further contemplate that all these methods are adaptable to high-throughput formats using robotic fluid dispensers, multi-well formats and fluorescent plate readers for the identification of serotonin transport modulators.

C. In Vivo Assays

In vivo assays are also contemplated in the present invention for secondary screening of variant SERT molecules for altered drug interaction. Such assays involve the use of various animal models, including transgenic animals that have been engineered to have specific defects, or carry markers that can be used to measure the ability of a candidate substance to reach and effect expression of a serotonin transporter in different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors or blockers of the serotonin transporter may be conducted using an animal model derived from any of these species.

In such assays, one or more candidate substance is administered to an animal, and the ability of the candidate substance(s) to alter one or more characteristics that are a result of serotonin function or activity, as compared to a similar animal not treated with the candidate substance(s), identifies an inhibitor or blocker. The characteristics may be any of those discussed above with regard to the function or activity of the serotonin neurotransmitter such as change in neurotransmission, change in the activity of some other downstream protein due to a change in neurotransmission, or instead a broader indication such as behavior of an animal, etc.

The present invention provides methods of screening for candidate substance that block or inhibit the serotonin transporter function or activity. In these embodiments, the present invention is directed to a method for determining the ability of a candidate substance to inhibit or block the serotonin transporter function, generally including the steps of: administering a candidate substance to the animal; and determining the ability of the candidate substance to change one or more characteristics of the serotonin transporter.

Treatment of these animals with candidate substance(s) will involve the administration of the substance, in an appropriate form, to the animal. Administration will be by any route that could be utilized for clinical or non-clinical purposes, including but not limited to oral, nasal, buccal, or even topical. Alternatively, administration may be by parenteral methods such as intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

1. In Vivo Microdialysis

Microdialysis may be used in the present invention to monitor interstitial fluid in various body organs with respect to local metabolic changes. This technique may also be experimentally applied in humans for measurements in adipose tissue. In the present invention, the release of serotonin in the mouse brain, in response to stimuli may be analyzed using this technique.

Microdialysis procedure involves the insertion through the guide cannula of a thin, needle-like perfusable probe (CMA/12.3 mm×0.5 mm) to a depth of 3 mm in striatum beyond the end of the guide. The probe is connected beforehand with tubing to a microinjection pump (CMA-/100). The probe may be perfused at 2 μl/min with Ringer's buffer (NaCl 147 mM; KCl 3.0 mM; CaCl₂ 1.2 mM; MgCl₂ 1.0 mM) containing 5.5 mM glucose, 0.2 mM L-ascorbate, and 1 μM neostigmine bromide at pH 7.4). To achieve stable baseline readings, microdialysis may be allowed to proceed for 90 minutes prior to the collection of fractions. Fractions (20 μl) may be obtained at 10 minute intervals over a 3 hour period using a refrigerated collector (CMA170 or 200). Baseline fractions may be collected, following the drug or combination of drugs to be tested, been administered to the animal. Upon completion of the collection, each mouse may be autopsied to determine accuracy of probe placement.

2. Behavioral Testing

Behavioral tests may be conducted as a follow on the SERT transport screens described above to further assess the efficacy of an given drug on a SERT variant. Such tests may include but are not limited to elevated plus-maze test, chronic mild stress test, forced swimming test, social defeat stress-induced anxiety test, or the light/dark test.

a. Elevated Plus-Maze Test in Mice

The apparatus may be based on that described by Pellow et al. (1985). In this procedure, the apparatus is elevated and contains two open and two enclosed arms, arranged so that the arms of the same type are opposite to each other. The apparatus is equipped with infrared beams and sensors capable of measuring arm activity for a given period of time. In addition, mice may be observed via video link by an observer located in an adjacent room. This arrangement allowed the recording of attempts at entry into open arms followed by avoidance responses, including stretched attend posture (the mouse stretches forward and retracts to original position). Tests may be performed 60 min after p.o. administration of the drugs.

b. Light/Dark Test in Mice

For this test, the apparatus may be based on that described by Belzung et al. (1989). For example, the apparatus may consist of two poly(vinyl chloride) boxes (20×20×14 cm), one of which is darkened. A desk lamp may be placed 20 cm above the lit box provided the room illumination. An opaque plastic tunnel (5×7×10 cm) may be used to separated the dark box from the illuminated one. The apparatus may be equipped with infrared beams capable of recording during a specific time period: (i) time spent by mice in the lit box, and (ii) number of tunnel crossings. Tests may be performed 30 min after i.p. administration of the drugs.

C. Forced Swimming Test in Mice

The forced swim test (FST) is widely used in the art for screening substances with a potential antidepressant effect. This procedure was originally described by Porsolt et al. (1977) however, modification may be made. Basically, the duration of immobility of the mice is measured for a given time period. The immobility observed by the FST is interpreted as “behavioral despair.”

3. Transgenic Animals

A transgenic animal of the present invention may involve an animal in which a variant serotonin transporter molecule is expressed temporally or spatially in a manner different than a non-transgenic animal. It is contemplated that the transgene, such as a gene encoding a serotonin transporter, may be expressed in a different tissue type or in a different amount or at a different time than the endogenously expressed version of the transgene. In addition, the invention contemplates creation of “knock-out” animals to eliminate endogenous SERT expression, as well as “knock-in” animal where an exogenous SERT replace endogenous SERT is one event.

In a general aspect, a transgenic animal is produced by the integration of a given transgene into the genome in a manner that permits the expression of the transgene, or by disrupting the wild-type gene, leading to a knockout of the wild-type gene. Methods for producing transgenic animals are generally described by Wagner and Hoppe (U.S. Pat. No. 4,873,191; which is incorporated herein by reference; Brinster et al. 1985; which is incorporated herein by reference in its entirety; and in Hogan, 1994; which is incorporated herein by reference in its entirety).

U.S. Pat. No. 5,639,457 is also incorporated herein by reference to supplement the present teaching regarding transgenic pig and rabbit production. U.S. Pat. Nos. 5,175,384; 5,175,385; 5,530,179, 5,625,125, 5,612,486 and 5,565,186 are also each incorporated herein by reference to similarly supplement the present teaching regarding transgenic mouse and rat production. Transgenic animals may be crossed with other transgenic animals or knockout animals to evaluate phenotype based on compound alterations in the genome.

As used herein, the term “transgene” means an exogenous gene introduced into a mouse through human intervention, e.g., by microinjection into a fertilized egg or by other methods known to those of average skill in the art. The term includes copies of the exogenous gene present in descendants of the mouse into which the exogenous gene was originally introduced. Likewise, the term “transgenic mouse” includes the original mouse into which the exogenous gene was introduced, as well as descendants of the original mouse so long as such descendants carry the transgene.

The transgenic animal of the invention may be produced by introducing transgenes into the germline of the animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor.

Introduction of the transgene into the embryo can be accomplished by any means known in the art such as, for example, microinjection, electroporation, or lipofection. For example, the serotonin transporter transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg may be incubated in vitro for varying amounts of time, or reimplanted into the surrogate host, or both. In vitro incubation to maturity is within the scope of this invention. One common method is to incubate the embryos in vitro for about 1-7 days, depending on the species, and then reimplant them into the surrogate host.

Retroviral infection can also be used to introduce transgene into a non-human animal. The developing non-human embryo can be cultured in vitro to the blastocyst stage. During this time, the blastomeres can be targets for retroviral infection (Jaenich, 1976). Efficient infection of the blastomeres is obtained by enzymatic treatment to remove the zona pellucida (Manipulating the Mouse Embryo, 1986). The viral vector system used to introduce the transgene is typically a replication-defective retrovirus carrying the transgene (Jahner et al., 1985; Van der Putten et al., 1985). Transfection is easily and efficiently obtained by culturing the blastomeres on a monolayer of virus-producing cells (Van der Putten, et al., 1985; Stewart et al., 1987). Alternatively, infection can be performed at a later stage. Virus or virus-producing cells can be injected into the blastocoele (Jahner et al., 1982). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al., 1982).

Embryonal stem cells (ES) may also be used for introducing transgenes. ES cells are obtained from pre-implantation embryos cultured in vitro and fused with embryos (Evans et al., 1981; Bradley et al., 1984; Gossler et al., 1986; and Robertson et al., 1986). Transgenes can be efficiently introduced into the ES cells by DNA transfection or by retrovirus-mediated transduction. Such transformed ES cells can thereafter be combined with blastocysts from a animal. The ES cells thereafter colonize the embryo and contribute to the germ line of the resulting chimeric animal (Jaenisch, 1988). ES cell are often used to create “knock in” or “knock out” animals.

A DNA fragment may be introduced into a mouse genome to produce a transgenic line of mice. The DNA fragment, usually a linear portion of a plasmid designed to express a gene under known genetic control elements, is microinjected into a pronucleus of a blastocyst or zygote. The injected cell develops following its introduction into the oviduct of pseudo-pregnant recipient female mice. If the DNA integrates into one of the chromosomes (it usually does so during the first few cell divisions of preimplantation development), then the transgenic founder mice are mosaic for the presence of the injected DNA. Founders produced in this way are very likely to have germ cells with the integrated transgene, and therefore will be able to transmit the integrated gene. In this way, transgenic lines of mice are produced, in which all cells of a transgenic mouse contain the transgene. The number of copies of the integrated DNA fragment can vary from one to several hundred, primarily arranged in a head-to-tail array.

The number of copies of the transgene constructs which are added to the cell is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. There is often an advantage to having more than one functioning copy of each of the inserted exogenous DNA sequences to enhance the phenotypic expression of the exogenous DNA sequences. For the purposes of this invention a zygote is essentially the formation of a diploid cell which is capable of developing into a complete organism. Generally, the a will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the cell or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the cell. The biological limit of the number and variety of DNA sequences will vary depending upon the particular cell and functions of the exogenous genetic material and will be readily apparent to one skilled in the art, because the genetic material, including the exogenous genetic material, of the resulting cell must be biologically capable of initiating and maintaining the differentiation and development of the zygote into a functional organism.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

### V. TREATMENT OF SERT-RELATED DISEASE STATES

A. Disease States

In other particular embodiments, the present invention provides a methods of treating a neurologic or psychiatric condition associated with BAT dysfunction comprising administering to a subject in need thereof a therapeutically effective amount of a p38 MAPK modulator, alone or in combination with a BAT (e.g., SERT or DAT) modulator. Neurologic or psychiatric conditions that may be treated using a candidate substance of the invention include, but are not limited to, obsessive compulsive disorders (OCDs), autism, generalized anxiety disorders, pathological aggression, schizophrenia, schizotypal personality disorder, psychosis, a schizoaffective disorder, manic type disorder, a bipolar affective disorder, a bipolar affective (mood) disorder with hypomania and major depression (BP-II), a unipolar affective disorder, unipolar major depressive disorder, dysthymic disorder, a phobia, a panic disorder, a somatization disorder, hypochondriasis, or an attention deficit disorder.

B. Diagnostics

In conjunction with other aspects of the invention, one may first screen an individual for their susceptibility to treatment with a given drug by identifying the existence of polymorphisms in the SERT gene and/or SERT protein. A variety of methods for assessing alterations in the structure of a SERT gene or protein are envisioned. Exemplary amino acid alterations that may be identified through either nucleic acid-based or protein-based diagnostics are provided in Table 5, below.

1. Nucleic Acid-Based Diagnostics

There are a large variety of techniques that can be used to assess small alterations in nucleic acids (e.g., SNPs), and more are being discovered each day. Such methods include but are not limited to, fluorescent in situ hybridization (FISH), DNA sequencing, PFGE analysis, Southern or Northern blotting, single-stranded conformation analysis (SSCA), primer extension, RNAse protection assay, allele-specific oligonucleotide (ASO), dot blot analysis, denaturing gradient gel electrophoresis, RFLP and PCR™-SSCP. The following is a very general discussion of a few of these methods and general techniques that can be used in accordance with the present invention.

a. RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even strains) from one another.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in SNP analysis requires that the SNP affect cleavage of at least one restriction enzyme site.

b. Primer Extension

The primer and no more than three NTPs may be combined with a polymerase and the target sequence, which serves as a template for amplification. By using less than all four NTPs, it is possible to omit one or more of the polymorphic nucleotides needed for incorporation at the polymorphic site. It is important for the practice of the present invention that the amplification be designed such that the omitted nucleotide(s) is(are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

A specific form of primer extension, developed by the inventor, can be found in U.S. Ser. No. 10/407,846, which is hereby specifically incorporated by reference.

C. Oligonucleotide Hybridization

Oligonucleotides may be designed to hybridize directly to a target site of interest. The most common form of such analysis is where oligonucleotides are arrayed on a chip or plate in a “microarray.” Microarrays comprise a plurality of oligos spatially distributed over, and stably associated with, the surface of a substantially planar substrate, e.g., biochips. Microarrays of oligonucleotides have been developed and find use in a variety of applications, such as screening and DNA sequencing.

In gene analysis with microarrays, an array of “probe” oligonucleotides is contacted with a nucleic acid sample of interest, i.e., target. Contact is carried out under hybridization conditions and unbound nucleic acid is then removed. The resultant pattern of hybridized nucleic acid provides information regarding the genetic profile of the sample tested. Methodologies of gene analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as. preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

d. Sequencing

DNA sequencing enables one to perform a thorough analysis of DNA because it provides the most basic information of all: the sequence of nucleotides. Maxam & Gilbert developed the first widely used sequencing methods—a “chemical cleavage protocol.” Shortly thereafter, Sanger designed a procedure similar to the natural process of DNA replication. Even though both teams shared the 1980 Nobel Prize, Sanger's method became the standard because of its practicality.

Sanger's method, which is also referred to as dideoxy sequencing or chain termination, is based on the use of dideoxynucleotides (ddNTP's) in addition to the normal nucleotides (NTP's) found in DNA. Dideoxynucleotides are essentially the same as nucleotides except they contain a hydrogen group on the 3′ carbon instead of a hydroxyl group (OH). These modified nucleotides, when integrated into a sequence, prevent the addition of further nucleotides. This occurs because a phosphodiester bond cannot form between the dideoxynucleotide and the next incoming nucleotide, and thus the DNA chain is terminated. Using this method, optionally coupled with amplification of the nucleic acid target, one can now rapidly sequence large numbers of target molecules, usually employing automated sequencing apparati. Such techniques are well known to those of skill in the art.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process. In various embodiments, the probes or primers are labeled with radioactive species (³²P, ¹⁴C, ³⁵S, ³H, or other label), with a fluorophore (rhodamine, fluorescein) or a chemillumiscent (luciferase).

e. Mass Spectrometry

By exploiting the intrinsic properties of mass and charge, mass spectrometry (MS) can resolved and confidently identified a wide variety of complex compounds. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000).

f. Hybridization

There are a variety of ways by which one can assess genetic profiles, and may of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

g. Detectable Labels

Various nucleic acids may be visualized in order to confirm their presence, quantity or sequence. In one embodiment, the primer is conjugated to a chromophore but may instead be radiolabeled or fluorometrically labeled. In another embodiment, the primer is conjugated to a binding partner that carries a detectable moiety, such as an antibody or biotin. In other embodiments, the primer incorporates a fluorescent dye or label. In yet other embodiments, the primer has a mass label that can be used to detect the molecule amplified. Other embodiments also contemplate the use of Taqman™ and Molecular Beacon probes. Alternatively, one or more of the dNTPs may be labeled with a radioisotope, a fluorophore, a chromophore, a dye or an enzyme. Also, chemicals whose properties change in the presence of DNA can be used for detection purposes. For example, the methods may involve staining of a gel with, or incorporation into the separation media, a fluorescent dye, such as ethidium bromide or Vistra Green, and visualization under an appropriate light source.

The choice of label incorporated into the products is dictated by the method used for analysis. When using capillary electrophoresis, microfluidic electrophoresis, HPLC, or LC separations, either incorporated or intercalated fluorescent dyes are used to label and detect the amplification products. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer no label is required because nucleic acids are detected directly.

In the case of radioactive isotopes, tritium, ¹⁴C and ³²P are used predominantly. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

h. Other Methods of Detecting Nucleic Acids

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference in its entirety.

i. Oligonucleotide Synthesis

Oligonucleotide synthesis is well known to those of skill in the art. Various mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference in its entirety. Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore, purifications are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligodeoxynucleotides (Gillam et al., 1978). Under controlled conditions, polynucleotide phosphorylase adds predominantly a single nucleotide to a short oligodeoxynucleotide. Chromatographic purification allows the desired single adduct to be obtained. At least a trimer is required to initiate the method of adding one base at a time, a primer that must be obtained by some other method. The polynucleotide phosphorylase method works and has the advantage that the procedures involved are familiar to most biochemists.

Solid-phase methods. The technology developed for the solid-phase synthesis of polypeptides has been applied after an, it has been possible to attach the initial nucleotide to solid support material has been attached by proceeding with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, 1993) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

j. Separation of Nucleic Acids

In certain embodiments, nucleic acid products are separated by agarose, agarose-acrylamide or polyacrylamide gel electrophoresis using standard methods (Sambrook et al., 1989). Separated products may be cut out and eluted from the gel for further manipulation. Using low melting point agarose gels, the skilled artisan my remove the separated band by heating the gel, followed by extraction of the nucleic acid.

Separation of nucleic acids may also be effected by chromatographic techniques known in the art. There are many kinds of chromatography that may be used in the practice of the present invention, including capillary adsorption, partition, ion-exchange, hydroxylapatite, molecular sieve, reverse-phase, column, paper, thin-layer, and gas chromatography as well as HPLC.

A number of the above separation platforms can be coupled to achieve separations based on two different properties. For example, some of the primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

k. Template Dependent Amplification

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989), incorporated herein by reference in its entirety.

2. Protein Based Diagnostics

The present invention may also utilize methods of examining protein structure to assess SERT variation and susceptibility to drug action. Such methods include determining SERT protein mass, determining SERT phosphorylation state, determining SERT glycosylation state, or determining SERT 5HT flux/surface density rations.

a. Immunologic Diagnosis

Anti-SERT antibodies may be used in accordance with the present invention in a variety of contexts. In on aspects, antibodies that identify a particular SERT variant may be used in an ELISA assay. For example, variant-specific antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting the same to a second antibody having specificity for a conserved epitope of SERT. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° C. to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 h at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

Anti-SERT antibody compositions will find use in immunoblot or Western blot analysis as well. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

b. SERT Phosphorylation Assays

SERT phosphorylation assays were performed as described in Ramamoorthy et al. (1998). Briefly, 293-hSERT cells are seeded on poly-D-lysine-coated 6-well plates at 5×10⁵ cells/well. After 48 h, monolayers are washed once in phosphate-free DMEM and incubated for 1 h at 37° C. Typically, cells are then incubated at 37° C. with the same medium containing 1 mCi/ml carrier-free [³²P]orthophosphate for 1 h to equilibrate the intracellular ATP pools with labeled phosphate. Effectors or vehicles are added to the medium, and the incubation is continued at 37° C. Kinase inhibitors are preincubated for 30 min prior to addition of kinase activators or phosphatase inhibitors for the times indicated. The adherent cells are washed three times with phosphate-buffered saline and lysed by the addition of 400 μl/well ice-cold modified radioimmunoprecipitation (RIPA, 1 0 mM Tris, 150 mM NaCl, 1 mM EDTA, 0.1% SDS, 1% Triton X-100, 1% sodium deoxycholate, pH 7.4) buffer containing protease (1 μM pepstatin A, 250 μM phenylmethylsulfonyl fluoride, 1 μg/ml leupeptin, 1 μg/ml aprotinin) and phosphatase inhibitors (10 mM sodium fluoride, 50 mM sodium pyrophosphate, and 1 μM okadaic acid) for 1 h at 4° C. with agitation. RIPA extracts were centrifuged at 20,000×g for 30 min at 4° C.

Protein content of supernatant is assessed using the DC protein assay (Bio-Rad) with bovine serum albumin as the standard. Protein content between wells and experiments showed <5% variability. Labeling with Trans³⁵S-label is carried out in Cys/Met-free DMEM as described previously (Qian et al., 1995; Melikian et al., 1994). Supernatants are precleared by the addition of 100 μl (3 mg) of Protein A-Sepharose beads for 1 h at 4° C. hSERT protein is immunoprecipitated overnight at 4° C. by the addition of SERT-specific antibody, CT-2 (10 μl of antisera) on end-over-end continuous mixing, followed by 1-h incubation with Protein A-Sepharose beads (3 mg in 100 μl in RIPA buffer) at 22° C. Additional experiments to test specificity are carried out with the hNET-specific antibody, preimmune serum, or a second SERT-specific serum. The immunoadsorbents are washed three times with ice-cold RIPA buffer prior to the addition to 50 μl of Laemmli sample buffer (62.5 mM Tris-HCl, pH 6.8, 20% glycerol, 2% SDS, 5% β-mercaptoethanol, and 0.01% bromphenol blue), incubated for 30 min at 22° C., and then resolved by SDS-PAGE (10%), with radiolabeled proteins detected by autoradiography or direct PhosphorImager (Molecular Dynamics) analysis. The relative amounts of ³²P incorporated into hSERT protein are estimated using ImageQuant software (Molecular Dynamics). Quantitation from digitized autoradiograms can be evaluated on multiple film exposures to ensure quantitation within the linear range of the film and gave identical results to estimations achieved with direct PhosphorImager quantitation.

C. SERT Glycosylation Assays

SERT glycosylation assays can be performed as set forth in Tate and Blakely (1994), Ramamoorthy et al. (1998) and Melikian et al. (1994, 1996).

### VI. PHARMACEUTICAL FORMULATIONS

The present invention also contemplates the administration of substance(s) as therapeutic agents (e.g., p38 MAPK agonists or antagonists) for the treatment of SERT-related neurological diseases. The substance(s) may be prepared in pharmaceutical compositions. Generally, this will entail preparing compositions that are essentially free of pyrogens, as well as other impurities that could be harmful to humans or animals.

Okadaic acid, calyculin A, fostriecin are PPA2 inhibitors and may be used in accordance with the present invention. Peptides DT-2 and DT-3 (YGRKKRRQRRRPP-LRK₅H-amide and RQIKIWFQNRRMKWKK-LRK₅H-amide), RKRARKE and RQIKIWFQNRRMKWKKLRKKKKKH are inhibitors of PKG. Other PKG modulators include Rp-Guanosine 3′,5′-cyclic monophosphorothioate, Rp-8-Bromo-Guanosine 3′,5′-cyclic monophosphorothioate, Rp-beta-Phenyl-1,N²-etheno-8-bromo-Guanosine 3′,5′-cyclic monophosphorothioate, [(9S,10R,12R)-2,3,9,10,11,12-hexahydro-10-methoxy-2,9-dimethyl-1-oxo-9,12-epoxy-1H-diindolo-[1,2,3-fg-3′,2′,1′-kl]pyrrolo[3,4-i][1,6]benzodiazocine-10-carboxylic acid methyl ester, 1-(5-Isoquinolinesulfonyl)-2-methylpiperazine, KT5823 (Axxora) and staurosporin. A large number of PDE-5 inhibitors are known, including sildenafil, zaprinast vardenafil and tadalafil (PDE-5). SERT inhibitors include sertraline, paroxetine, citalopram, and fluoxetine, while anisomycin is a know SERT activator. NET inhibitors include mazindol, reboxetine and atomoxetine, despiramine and nisoxetine.

One will generally desire to employ appropriate salts and buffers. Aqueous compositions of the present invention comprise an effective amount of the neurotransmitter transporter modulator dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Supplementary active ingredients also can be incorporated into the compositions.

Administration of these compositions according to the present invention will be via any common route so long as the target tissue is available via that route. This includes administration may be by systemic or parenteral methods including intravenous injection, intraspinal injection, intracerebral, intradermal, subcutaneous, intramuscular, intraperitoneal methods. Depending on the nature of the modulator administration may also be via oral, nasal, buccal, rectal, vaginal or topical. Such compositions would normally be administered as pharmaceutically acceptable compositions, described supra.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The composition may be formulated as a “unit dose.” For example, one unit dose could be dissolved in 1 ml of isotonic NaCl solution and either added to 1000 ml of hypodermoclysis fluid or injected at the proposed site of infusion, (see for example, “Remington's Pharmaceutical Sciences,” 15^{th} Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

DNA Constructs. The full-length cDNA encoding hSERT in the mammalian expression vector pcDNA3.1 (Invitrogen) has been previously described (Qian et al., 1997). Mutations in hSERT were producing using the QuikChange mutagenesis kit (Stratagene). All mutations were confirmed by fluorescent dideoxynucleotide sequencing (Center for Molecular Neuroscience Neurogenomics Core).

Transfection and Transport Studies. HeLa cells, maintained at 37° C. in a 5% CO2 humidified incubator, were grown in complete medium (Dulbecco's Modified Eagle's Medium (DMEM, Invitrogen), 10% fetal bovine serum, 2 mM L-glutamine, 100 units/ml penicillin, and 100 μg/ml streptomycin). Transfections (1 μg DNA/500,000 cells/6-well plate or 0.05 μg/10,000 cells/24-well plate) were performed using Fugene6 reagent (Roche) in Opti-MEM I (Invitrogen) as suggested by the manufacturer. Transfected cells were cultured as above for 36 hr prior to 5-HT transport and biochemical assays.

Transport, Binding, Biotinylation and Phosphorylation Studies. Transport of [³H]5-HT (5-hydroxy[³H]tryptamine trifluoroacetate, Amersham Biosciences, 20 nM final conc) was conducted in a total assay volume of 500 μl Krebs-Ringer-HEPES (KRH) assay buffer containing 130 mM NaCl, 1.3 mM KCl, 2.2 mM CaCl₂, 1.2 mM MgSO₄, 1.2 mM KH₂PO₄, 1.8 g/L glucose, and 10 mM HEPES, pH 7.4, as previously described (Zhu et al., 2004a,b), defining specific 5-HT uptake using 10 μM paroxetine. For quantitative assessment of SERT total and surface density, the inventors measured [¹²⁵I]RTI-55 binding(5 nM) to intact cells on ice, utilizing either paroxetine (1 μM) or 5-HT (100 μM) as displacer (Zhu et al., 2004a,b). To establish levels and biosynthetic progression of hSERT protein produced from mutant cDNAs, HeLa cells were plated in 6-well dishes at 500,000 cells per well and transfected 12 h later. Twenty four hours after transfection, whole cell detergent extracts were blotted for hSERT (mAb Technologies, Inc., 1:1000) using enhanced chemiluminescence (ECL, Amersham) detection. Altered density of SERT surface proteins was validated using immunoblotting of biotinylated whole cell extracts produced using the lysine-directed, membrane-impermeant biotinylating reagent sulfo-NHS—SS-biotin (Pierce) (Qian et al., 1997). Phosphorylation of hSERT and Gly56Ala variants in transfected HeLa cells was examined as previously described (Cohen et al., 2004) using 100 μM 8BrcGMP (60 min) as stimulus. Specificity of labeling was verified using parallel cultures transfected with pcDNA3.

Genotyping and Lymphocyte Studies. Lymphocyte lines were derived from autistic pedigrees recruited by Susan E. Folstein (Johns Hopkins Univ), from the Autism Genetics Resource Exchange (AGRE) collection (www.agre.org/), and from the NIMH Human Genetics Initiative Repository (www.nimhgenetics.org/) at Rutgers University. DNA from lymphoblastoid cell lines was genotyped via TaqMan-based allelic discrimination, using an Applied Biosystems (Foster City, Calif.) Assay-by-Design and independently confirmed by PCR and direct sequence analysis. Genotyped lymphocytes were cultured in suspension in RPMI 1640 medium, supplemented with 15% fetal bovine serum, 2 mM L-glutamine, 100 units/ml penicillin, and 100 μg/ml streptomycin) at 37° C. in a humidified incubator at 5% CO2 and maintained in uniform growth conditions. Lymphocyte were pelleted at 1500 rpm, 5 min and washed with KRH assay buffer. A total of 1×10⁶ cells in triplicate were pre-warmed (37° C.) in a shaking water bath (10 min) in 12×75 polypropylene tubes in KRH buffer containing 100 μM pargyline and 100 μM ascorbic acid+/−modifiers. After 5-min incubation with [³H]5-HT (20 nM)+/−10 μM paroxetine at 37° C., uptake assays were terminated by immersion on ice, and uptake in pelleted, SDS (1%)-extracted cells quantitated by scintillation spectrometry.

### Example 2

### Results

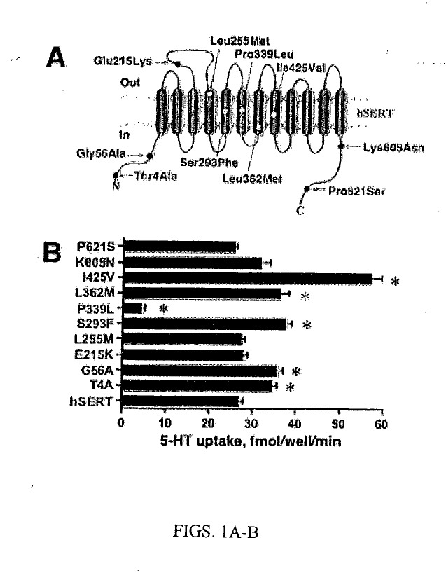

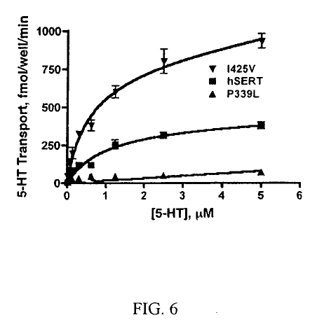

The location of ten identified hSERT coding variants is described in FIG. 1 A (see also Tables 1 and 2). The reference hSERT cDNA and each hSERT variant were separately transfected into HeLa cells and 5-HT transport activity assessed as described in Methods. As shown in FIG. 1B, five variants (Thr4Ala, Gly56Ala, Ser293Phe, Leu362Met, and Ile425Val) displayed enhanced 5-HT transport activity relative to hSERT and one variant (Pro339Leu) displayed markedly reduced uptake activity. These variations persisted across multiple plasmid preparations and thus appear to arise from intrinsic differences in protein abundance, transport rates or both. More detailed kinetic studies were pursued for the two variants displaying the largest shifts in transport activity, Ile425Val and Pro339Leu. Kinetic analysis of Ile425Val revealed significant changes in 5-HT Vₘₐₓ (190+/−28% of hSERT, p<0.05) and Kₘ (0.56+/−0.20 μM vs hSERT 1.00+/−0.47 μM, p<0.05) (see also FIG. 6). With Pro339Leu, 5-HT Vₘₐₓ was significantly reduced (3.0+/−1.2% of hSERT, p<0.05). The activity of Pro339Leu was too low to allow the 5-HT Kₘ to be reliably determined.

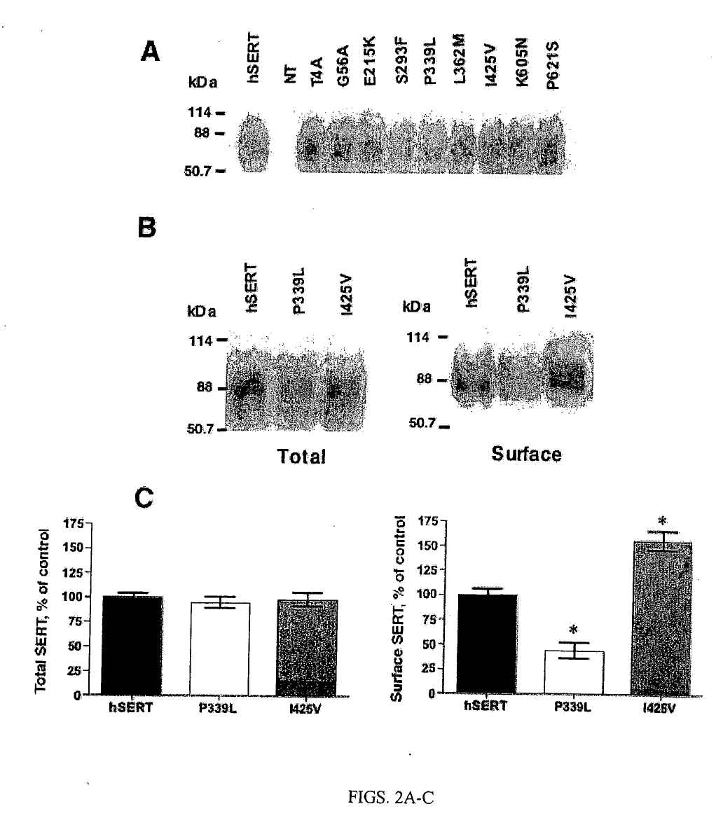

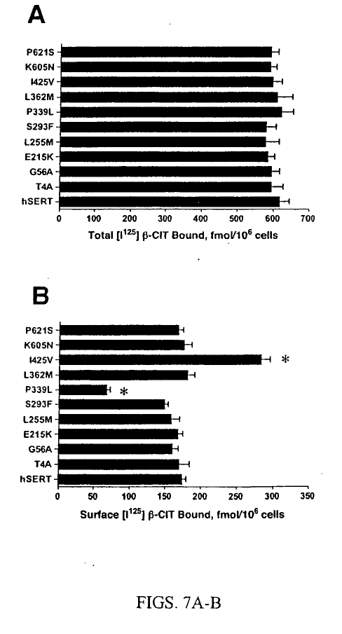

To establish a physical basis for the altered transport activities, immunoblots of transfected HeLa cell extracts were obtained. As shown in FIG. 2A, hSERT and almost all variants produced comparable levels of the 80 kDa protein that is characteristic of mature N-glycosylated hSERT protein (Qian et al., 1997). Biotinylation studies of the two variants bearing the largest changes in 5HT uptake, Pro339Val and Ile425Val, revealed a significant reduction and elevation, respectively, of surface protein, paralleling shifts in 5-HT transport activity (FIGS. 2B-C). Total [¹²⁵I]RTI-55 binding, defined with the hydrophobic displacer paroxetine, did not demonstrate differences between hSERT and hSERT variants (FIG. 7A). Surface SERT density, as defined by 5-HT displacement, was significantly depressed for Pro339Val and elevated for Ile425Val (FIG. 7B), but unchanged for the other variants. In contrast, Thr4Ala, Gly56Ala, Ser293Phe, and Leu362Met variants also display enhanced basal transport activity (30-50%) that could not be explained by enhanced surface density. The inventors also evaluated whether variant SERTs retained normal antagonist sensitivities. Several variants demonstrated altered sensitivity to either r/s-fluoxetine, r/s-citalopram or cocaine (Table 5). Most prominently, the inventors observed a 10-fold shift in cocaine potency with Pro339Leu,. accompanied by a significant though less substantial loss of potency for citalopram and fluoxetine.

TABLE 5
Pharmacological Sensitivities of Human SERT Coding Variants

| | | | |
|---|---|---|---|
| hSERT | 3.8 ± 1.4 | 21 ± 7 | 345 ± 57 |
| T4A | 4.7 ± 1.9 | 46 ± 16 | 468 ± 22 |
| G56A | 8.9 ± 2.8 | 61 ± 24 | 2158 ± 394* |
| E215K | 5.2 ± 2.7 | 28 ± 13 | 543 ± 149 |
| L255M | 12.3 ± 1.2* | 68 ± 6 | 1852 ± 41* |
| S293F | 11.1 ± 4.5 | 40 ± 15 | 1603 ± 398* |
| P339L | 13.4 ± 5.3* | 132 ± 72* | 3787 ± 981* |
| L362M | 6.9 ± 3.1 | 46 ± 13 | 1700 ± 665* |
| I425V | 16.5 ± 6.4* | 23 ± 10 | 1188 ± 383 |
| K605N | 5.7 ± 2.2 | 37 ± 15 | 1567 ± 480* |
| P621S | 3.6 ± 1.7 | 29 ± 5 | 802 ± 232 |

Ki values (nM +/− SEM) were determined from three or more separate SERT uptake inhibition assays.
*p < 0.05, One-way ANOVA with a post hoc Dunnett's test comparing variant to hSERT for the same compound.

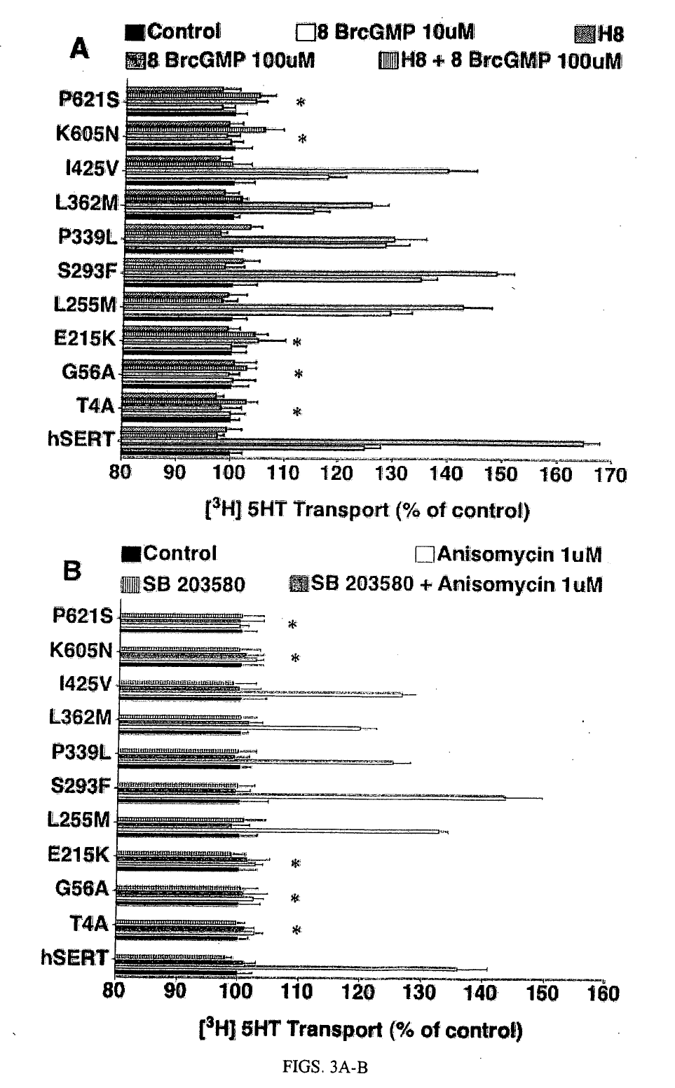

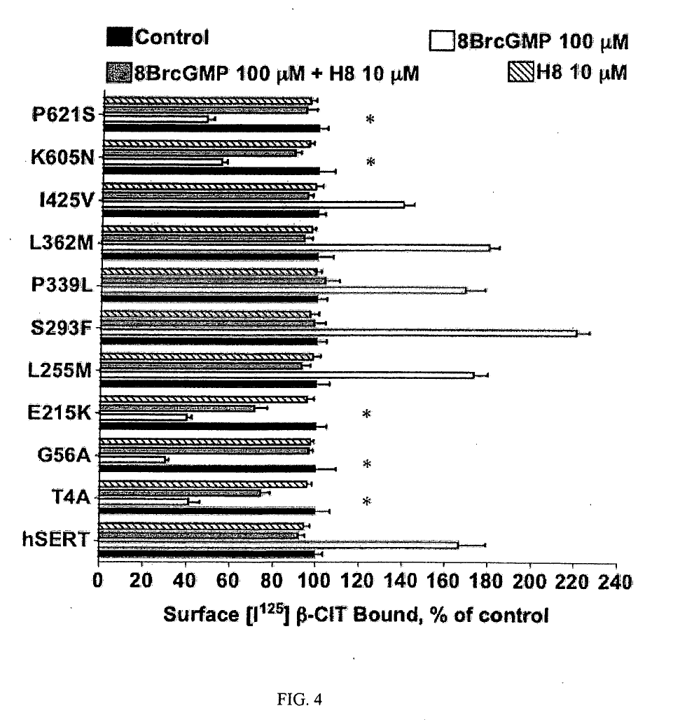

cGMP-linked pathways enhance SERT activity in native (Zhu et al., 2004a,b; Miller and Hoffman, 1994; Launay et al., 1994) and transfected (Zhu et al., 2004a,b; Kilic et al., 2003; Zhu et al., 2004a,b) cells. Similarly, when the inventors treated hSERT expressing HeLa cells with 8BrcGMP (10-100 μM, 10 min), they achieved a dose-dependent stimulation of 5-HT transport activity that peaked at a 50-70% increase at 100 μM 8BrcGMP, and which could be completely antagonized by coincubation with the PKG antagonist H8 (10 μM) (FIG. 3A). Parallel changes in [¹²⁵I]RTI-55 surface binding support increased surface trafficking triggered by the PKG pathway (FIG. 4). For variants Leu255Met, Ser293Phe, Pro339Leu, Leu362Met, and Ile425Met, 8BrcGMP triggered a dose-dependent, H8-sensitive stimulation of SERT activity comparable to hSERT. In contrast, Thr4Ala, Gly56Ala, Glu215Lys, Lys605Asn, and Pro621Ser were completely insensitive to 8BrcGMP application. The hSERT variants that responded with uptake increases also demonstrated elevated [¹²⁵I]RTI-55 surface binding. Remarkably, the five hSERT variants that failed to elicit uptake increases following 8BrcGMP treatments actually demonstrated a reduction in [¹²⁵I]RTI-55 surface binding (FIG. 4). These reductions in surface density were still specific as they could be completely blocked by H8. 8BrcGMP and H8 had no effects on total binding as assessed in parallel assays using paroxetine as the displacer (data not shown).

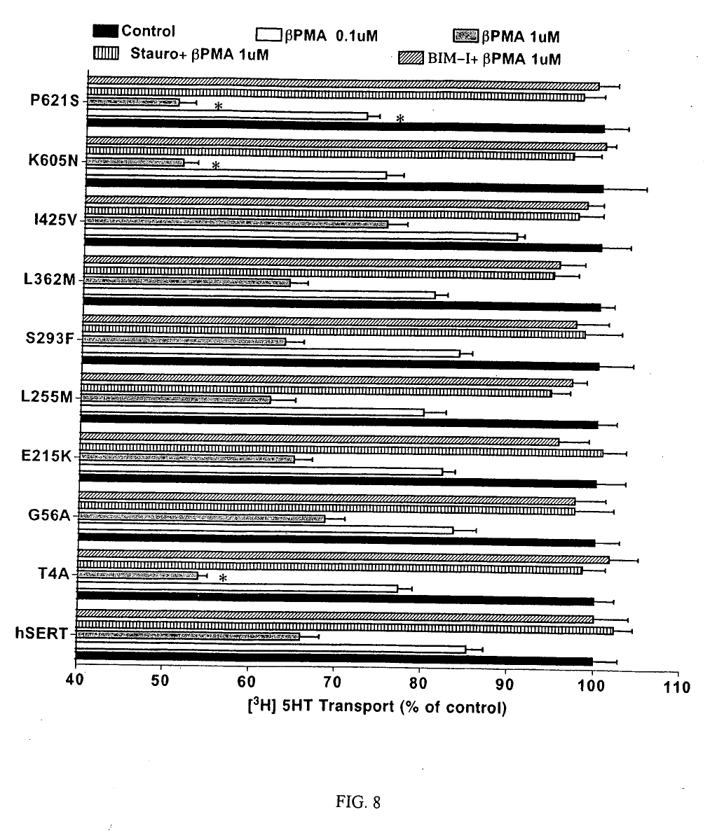

SERTs are known to be rapidly internalized by phorbol ester treatments, effects that are blocked by PKC antagonists (Ramamoorthy et al., 1998). Thus, the inventors treated transfected cells with the phorbol ester β-PMA and monitored changes in 5-HT transport activity. As expected hSERT expressed transiently in HeLa cells displays an ˜40% downregulation after a 15 min treatment with 10 μM β-PMA, downregulation blocked by the PKC antagonist bisindolylmaleimide (BIM, 1 μM) (FIG. 8). In contrast to findings with hSERT variants for 8BrcGMP treatments, each of the hSERT variants displayed downregulation following β-PMA treatments equal to or slightly greater (Thr4Ala, Lys605Asn and Pro621 Ser) that seen for hSERT.

In studies of adenosine receptor and PKG-linked upregulation of SERT, the inventors discovered that enhanced SERT activity requires activated p38 MAPK (Zhu et al., 2004a,b). More recent studies reveal that direct p38 MAPK activators such as anisomycin trigger a rapid, trafficking-independent upregulation of hSERT (Zhu et al., 2005). The inventors treated hSERT transfected cells with 1 μM anisomycin for 10 min prior to 5-HT transport assays and as previously found, achieved a 40-50% stimulation of uptake activity (FIG. 3B). Just as with 8BrcGMP treatments, variants Leu255Met, Ser293Phe, Pro339Leu, Leu362Met, and Ile425Val each responded to anisomycin treatment comparable to hSERT with increased activity blocked by co-treatments with SB203580. Neither the uptake stimulation of hSERT nor the upregulation achieved with these five variants was accompanied by changes in total or surface [¹²⁵I]RTI-55 binding (data not shown), consistent with a trafficking-independent mode of action of the p38 MAPK pathway (Zhu et al., 2004a,b; Zhu et al., 2005). Remarkably, when the five hSERT variants lacking 8BrcGMP sensitivity, Thr4Ala, Gly56Ala, Glu215Lys, Lys605Asn, and Pro621Ser, were tested with anisomycin, no uptake stimulation was observed.

Of the variants studied, only one, Gly56Ala, is found at frequencies sufficient to permit identification of subjects carrying modified alleles. The inventors genotyped a large collection of 340 autism families possessing in many cases banked, EBV-transformed lymphocytes, since SERT is natively expressed in lymphocytes (Lesch et al., 1996) and because the 17q11.2 region harboring the SERT gene demonstrated linkage in autism families (McCauley et al., 2004). They found the 56Ala allele at a frequency of 1.1% in all families, but this increased to 2.3% in 120 families most contributing to linkage. This frequency represents a significant difference (χ2=9.94, df=1, P=0.0016) between our sample and a separately collected, non-clinical sample (21). Importantly, the inventors identified two probands bearing a homozygous Ala56 genotype as well as multiple subjects carrying heterozygous genotypes.

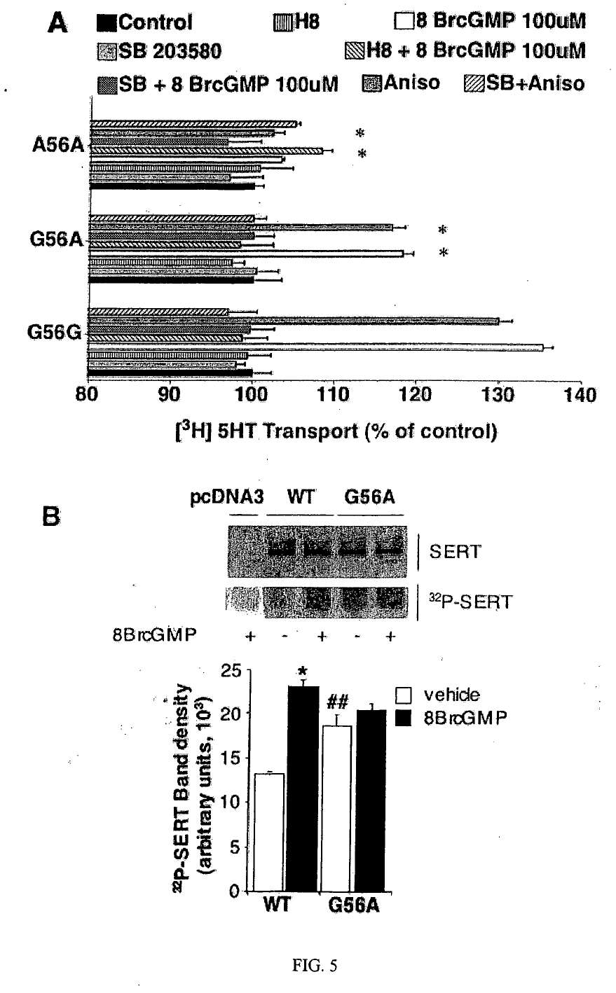

As seen with transfected cells, lymphocytes homozygous for the Gly variant (identical to reference hSERT) provided robust 8BrcGMP stimulation of 5-HT transport, stimulation that is sensitive to H8 (FIG. 5A). Additionally, anisomycin stimulated uptake activity and this stimulation was sensitive to SB203580. In contrast, the Ala56 homozygous lines lacked sensitivity to either 8BrcGMP or anisomycin. The Gly56Ala heterozygous cells displayed intermediate sensitivity to these agents, consistent with a gene dosage-dependent impact on regulation. As detected in transfected cells, the inventors found that all three genotypes displayed a similar degree of downregulation following β-PMA treatments (data not shown). SERT is phosphorylated under basal conditions and phosphorylation can be significantly elevated following PKG activation (Pritchard, 2001). To examine whether the loss of regulation exhibited by the Gly56Ala variant might derive from changes in its ability to receive regulatory phosphorylation, the inventors performed in situ phosphorylation studies, immunoprecipitating SERT proteins after stimulation of [³²P]pre-labeled cells with 8BrcGMP. In hSERT-transfected HeLa cells, 8BrcGMP (100 μM, 60 min) triggered an ˜80% elevation of basal phosphorylation (FIG. 5B). In contrast, Gly56Ala transfected cells exhibited significantly elevated basal phosphorylation levels and could not be further phosphorylated by 8BrcGMP treatments. Western blotting of cell extracts revealed no differences in total hSERT protein levels.

### Example 3

### Materials and Methods

Reagents and Constructs. Anisomycin, hydrogen peroxide (H₂O₂), fostriecin, calyculin A, curcumin, 1H-(1,2,4)-oxadiazolo[4,3-a]quinoxalin-1-one (ODQ), NECA, N-[2-(methylamino)ethy]-5-isoquinoline sulfonamide (H8), paroxetine, and desipramine were purchased from Sigma. GBR 12935 dihydrochloride is a product of Tocris (Ellisville, Mo.). SB203580 and SB202190 were obtained from Alexis Biochemicals (San Diego, Calif.). SB202474 dihydrochloride was purchased from Calbiochem. [2-(Trimethylammonium)ethyl]methanethiosulfonate (MTSET) was obtained from Toronto Research Chemicals Inc. (North York, Canada). [³H]-5-HT (5-hydroxy[³H]tryptamine trifluoroacetate; 102 Ci/mmol) was purchased from Amersham Biosciences; (3β-(4-iodophenyl)-tropane-2β-carboxylic acid methylester tartrate ([¹²⁵I]RTI-55); 2200 Ci/mmol) was purchased from PerkinElmer Life Sciences. Trypsin-EDTA, glutamine, and ampicillin/streptomycin were purchased from Invitrogen; modified Eagle's medium (MEM) and Dulbecco's MEM were derived from Invitrogen reagents and prepared in the Vanderbilt Media Core. Membrane-permeant PKG-inhibitory peptide DT-2 was synthesized as previously described (Dostmann et al., 2000). hSERT (Ramamoorthy et al., 1993), hNET (Pacholczyk et al., 1991), and hDAT (Giros et al., 1992) cDNAs have been described. hDAT was a gift from Dr. Marc Caron (Duke University, Durham, N.C.). Anti-total and phosphorylated p38 MAPK antibodies were purchased from Cell Signaling (La Jolla, Calif.). siGENOME™ SMARTpool® siRNA for rat p38 MAPK was a product of Dharmacon (Chicago, Ill.). Antibodies to β-actin were obtained from Sigma.

Cell Culture and Transfection. RBL-2H3 cells (ATCC, Manassas, Va.) were maintained at 37° C. in MEM containing 15% fetal bovine serum (Invitrogen), 1% L-glutamine (L-Gln), 100 IU/ml penicillin, and 100 μg/ml streptomycin. RN46A cells (provided by Dr. Whittemore, University of Miami School of Medicine, Miami, Fla.) were cultured at 33° C. with Dulbecco's MEM/F-12 (1:1 in volume) containing 250 mg/liter G418, 10% fetal bovine serum, 1% L-Gln, and 100 μg/ml penicillin/streptomycin (Eaton and Whittemore, 1995). CHO cells (ATCC, Manassas, Va.) were maintained at 37° C. in Dulbecco's MEM containing 10% fetal bovine serum, 1% L-Glu, 100 IU/ml penicillin, and 100 μg/ml streptomycin. For comparison of the response of monoamine transporters to p38 MAPK activation, CHO cells were transfected with cDNAs for hSERT, hDAT, or hNET (100 ng/well for a 24-well plate). Transfections of CHO cells were performed using TransIT-LT1 Transfection Reagent (Mirus, Madison, Wis.) according to the manufacturer's protocol (0.3 μl/well). hSERT, hDAT, or hNET cDNAs were preincubated with the reagent at ambient temperature for 30 min before being added to plated CHO cells. Cells were cultured for 24 h after transfection. The transfection mixture was removed just prior to assay.

5-HT Transport Assays. [³H]5-HT transport activity was assayed as described previously (Ramamoorthy et al., 1998; Zhu et al., 2004a,b). Briefly, RBL-2H3 cells were seeded in 24-well plates (40,000 cells/well) 24 h prior to the 5-HT uptake assay. CHO cells were plated at 20,000 cells/well 16-24 h before transfection, and the uptake assay was performed 24 h following the transfection. RN46A cells were plated at 200,000 cells/well 24 h before the 5-HT uptake assay. Medium was removed by aspiration, and cells were washed once with Krebs-Ringer HEPES (KRH) buffer containing 130 mM NaCl, 1.3 mM KCl, 2.2 mM CaCl2, 1.2 mM MgSO4, 1.2 mM KH2PO4, 1.8 g/liter glucose, 10 mM HEPES, pH 7.4. Cells were incubated in triplicate at 37° C. in KRH buffer (0.2 ml/well) containing 100 μM pargyline, 100 μM L-ascorbic acid, and/or 1.0 mM tropolone (Sigma), with or without modifiers. After a 10-min incubation with [³H]5-HT (100 nM for RBL-2H3 and RN46A cells, 20 nM for SERT-transfected CHO cells), [³H]DA (50 nM), or [³H]NE (50 nM) at 37° C., buffer was aspirated, and the cells were washed three times with ice-cold KRH buffer. Cells were solubilized with 0.5 ml of Microscint 20 (PerkinElmer Life Sciences), and tritium-labeled monoamine accumulation was quantitated using a TopCount plate scintillation counter (PerkinElmer Life Sciences). Specific 5-HT, DA, and NE uptake was determined by subtracting the amount of [³H]5-HT, [³H]DA, and [³H]NE accumulated in the presence of 10 μM paroxetine, GBR 12935, and desipramine, respectively. 5-HT saturation kinetics in RBL-2H3 and RN46A cells were defined as described for standard transport assays, using varying concentrations of [³H]5-HT (brought to 0.5 Ci/mmol using unlabeled 5-HT), and again, nonspecific uptake was defined with 10 μM paroxetine. For studies seeking to inactivate surface SERTs in RBL-2H3 cells, the inventors treated cells with the membrane-impermeant, cysteine-specific alkylating reagent MTSET (Chen et al., 1997), as previously described (Zhu et al., 2004a,b). Cells were treated either with vehicle or MTSET (10 mM) for 10 min on ice prior to washing, and cells were then re-equilibrated in normal medium at 37° C. with anisomycin-stimulated 5-HT transport determined as described above.

Platelet Isolation and Transport Assays. Human platelet-rich plasma was obtained from the American Red Cross. Approximately 2×10⁹ platelets were incubated with the appropriate pharmacological reagents for 10 min at 37° C. and collected by centrifugation at 2000 rpm for 30 s. Platelets were then washed twice with 1×PBS and resuspended in KRH buffer containing 130 mM NaCl, 1.3 mM KCl, 2.2 mM CaCl₂, 1.2 mM MgSO4, 1.2 mM KH2PO4, 1.8 g/liter glucose, 10 mM HEPES, 100 μM pargyline, 100 μM ascorbic acid, and 1 mM tropolone. After a 5-min incubation with 50 nM [3H]5-HT, uptake was terminated by filtration through GF/B Whatman paper. Filters were washed three times with ice-cold KRH buffer, immersed in scintillation liquid for 8 h, and counted by scintillation spectrometry. The counts obtained from the filtered samples were corrected for the nonspecific binding/uptake obtained using parallel samples incubated with paroxetine (1 μM). Studies were performed in duplicate and replicated at least three times.

[¹²⁵I]RTI-55 Binding Assays. To assess SERT surface density, the inventors quantitated the binding of the high affinity cocaine analog [¹²⁵I]RTI-55 (5 nM) to intact cells at 4° C. for 45 min in PBS/CM buffer (phosphate buffered saline, pH 7.4, with 0.1 mM CaCl2, 1.0 mM MgCl₂) in the presence or absence of a membrane-permeant (1 μM paroxetine) or membrane-impermeant (100 μM 5-HT) displacer, defining total and surface-specific binding, respectively (Zhu et al., 2004a,b). Binding was terminated by two rapid washes with ice-cold PBS/CM. Cells were solubilized with 1% SDS, and [¹²⁵I]RTI-55 bound was quantified using a Gamma 4000 counter (Beckman Instruments). Assays to assess 5-HT potency for inhibition of [¹²⁵I]RTI-55 binding were performed as described above, except with the inclusion of varying concentrations of unlabeled 5-HT.

Assays of p38 MAPK Activation. To monitor activation of p38 MAPK by anisomycin in intact cells, RBL-2H3 cells were seeded in 96-well plates (10⁵ cells/well) and cultured in MEM containing 15% fetal bovine serum in a 37° C. incubator with 5% CO₂ for 16-24 h. Medium was removed by aspiration, and cells were washed once with KRH buffer and then incubated in triplicate at 37° C. in KRH buffer with or without p38 MAPK activators for 5-10 min. Assay buffer was removed, and cells were immediately fixed with fresh 4% formaldehyde (Sigma) in PBS for 20 min at room temperature and washed four times with 1×PBS containing 0.1% Triton X-100 (Sigma) (5 min per wash) prior to 1 h of blocking with Odyssey blocking buffer (Li-Cor Biosciences, Lincoln, Nebr.). RBL-2H3 cells were subsequently probed with dual phosphorylated (Thr(P) and Tyr(P)) p38 MAPK polyclonal antibody (Cell Signaling; 1:400) in Odyssey blocking buffer with gentle mixing for 2 h at room temperature. Cells were then washed four times with 1×PBS containing 0.1% Tween 20 (Sigma) for 5 min. Bound antibody was detected with fluorescence-labeled secondary antibody-Alexa Fluor® 680 (avoiding exposure to light; 1:200 in Odyssey Blocking Buffer, Molecular Probes, Inc., Eugene, Oreg.) for 1 h. After four washes with 1×PBS, 0.1% Tween 20, the plate was scanned, and the captured image of the signal was processed and quantified with the Odyssey™ Infrared Imaging System (Li-Cor Biosciences). For UV radiation to activate p38 MAPK, plates seeded with cells were placed in a UV source (UV Stratalinker 1800, Stratagene, La Jolla, Calif.) that was preset to deliver 4×10⁴ μJ/cm² at room temperature.

Assay for p38 MAPK Suppression by RNA Interference. To further evaluate the role of p38 MAPK in anisomycin-stimulated SERT activity, RBL-2H3 cells (seeded at 4×10⁴ cells/well in a 24-well plate) were transfected with 100 nM siRNA SMARTpool® for rat p38 MAPK-α by using TransIT-LT1 transfection reagent (Mirus, Madison, Wis.) according to the manufacturer's protocol. SERT activity was tested 48 h after transfection, and p38 MAPK protein levels were assessed using anti-p38 MAPK antibodies. As controls, cell culture medium alone or/and transfection reagent (Trans-IT) without siRNA was tested in parallel with the siRNA transfection. Anti-β-actin antibodies were used to reprobe the membrane for equal blot loading.

Statistical Analyses. Statistical analyses, comparing base-line and compound-modified uptake, antagonist binding, or p38 MAPK activation, were performed with GraphPad Prism (GraphPad, San Diego, Calif.) using one- and two-way analyses of variance (ANOVA) with subsequent planned comparisons (Dunnett, Bonferroni) as well as t tests as noted in the legends to FIGS. 5 and 6. Saturation kinetic and competition binding data were fit using GraphPad Prism (rectangular hyperbola, one-site binding isotherm).

### Example 4

### Results

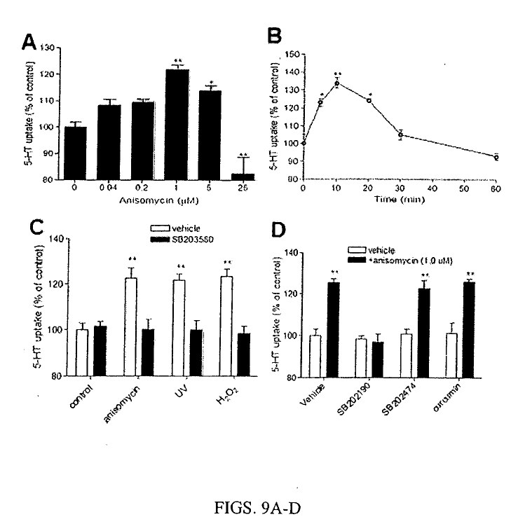

Stimulation of 5-HT Uptake in RBL-2H₃Cells by p38 MAPK Activators. The inventors previously demonstrated that AR agonists such as NECA rapidly augment p38 MAPK phosphorylation and elevate 5-HT uptake in RBL-2H3 cells in an SB203580-sensitive manner (Zhu et al., 2004a,b). Similar findings were obtained with AR/hSERT co-transfected CHO cells. These results, as well as the novelty of drug-modulated SERT catalytic function, warranted a more in depth evaluation of p38 MAPK-dependent SERT stimulation. To accomplish this, the inventors first explored the effects of the p38 MAPK activator, anisomycin (Zhang et al., 1997), on 5-HT uptake in RBL-2H3 cells. In these experiments (FIGS. 9A and 9B), the inventors found that anisomycin exerts a dose- and time-dependent stimulation of 5-HT transport activity. At 1 μm, anisomycin stimulation of 5-HT transport was transient, peaking at 10 min and declining to basal levels by 30 min. Using a 10-min pretreatment, anisomycin stimulation peaked at 1 μm and became inhibitory at 25 μm, effects that could represent toxicity, although this was not pursued further. Consistent with previous findings (Zhu et al., 2004a,b), the effects of anisomycin on 5-HT uptake were blocked by preincubation with the specific p38 MAPK inhibitor, SB203580 (FIG. 9C). Moreover, the effects of anisomycin could be mirrored by other p38 MAPK activators. Reactive oxygen species, such as H₂O₂, and UV radiation are known to trigger p38 MAPK activation in a wide range of cells (Moriguchi et al., 1996; New et al., 1998). Both H₂O₂ (20 μm) and UV light (4×10⁴ μJ/cm²) stimulated 5-HT transport to a level comparable with 1 μm anisomycin, and as with anisomycin, these effects were abolished by SB203580 (FIG. 9C). Additionally, other potent p38 MAPK inhibitors, SB202190 and PD169316 (data not shown), also blocked anisomycin stimulation of SERT, whereas the inactive analog, SB202474, and c-Jun NH₂— terminal kinase inhibitor, curcumin, lacked antagonistic activity (FIG. 9D). PD98059 (10 μm), a selective inhibitor of extracellular signal-regulated kinase ½, however, failed to block anisomycin-stimulated SERT activity (data not shown).

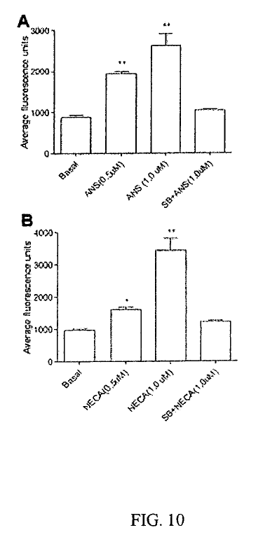

p38 MAPK must be dually phosphorylated to achieve catalytic activation (New et al., 1998). To verify that our anisomycin treatments of RBL-2H3 cells trigger p38 MAPK activation, the inventors monitored the dual phosphorylation status of the kinase monitoring total and phosphor-specific p38 MAPK antibodies using an In-Cell Western format. NECA and 8-bromo-cGMP (data not shown) were used as positive controls, since the inventors previously demonstrated these two agents activate p38 MAPK, with no change in total p38 MAPK (Zhu et al., 2004a,b). In the experiments shown, RBL-2H3 cells were treated with vehicle, NECA, or anisomycin (0.5 and 1.0 μm) for 5 min in KRH assay buffer in the absence/presence of the p38 MAPK inhibitor, SB203580. Similar to NECA, anisomycin stimulates a 2-3-fold increase in phosphorylated p38 MAPK, effects that are blocked by pretreatment with SB203580 (FIGS. 10A and 10B). The level of stimulation by NECA and anisomycin was comparable to stimulation achieved with 8-bromo-cGMP. No changes were observed in total p38 MAPK levels (data not shown).

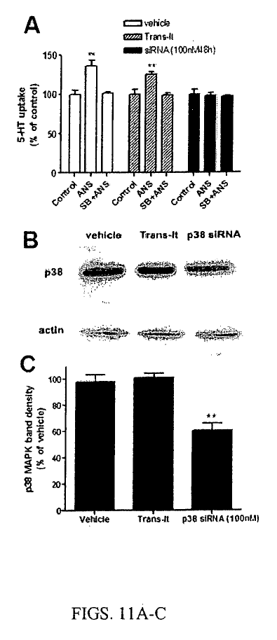

In order to validate a role for p38 MAPK in anisomycin triggered SERT up-regulation and to gain insight into specific p3 8 MAPK isoforms involved in SERT modulation, the inventors examined the impact of siRNA-mediated suppression of p38 MAPK-A on anisomycin stimulation. Treatment of RBL-2H3 cells with p38 MAPK-A siRNA (100 nM, 48 h) abolished the stimulation of 5-HT transport activity (FIG. 11A). Cells treated with transfection reagents alone retained up-regulation. Western blot analyses confirmed a down-regulation of p38 MAPK protein by these treatments (FIGS. 11B and 11C).

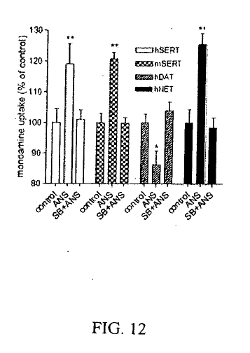

Anisomycin Induces 5-HT Transport in SERT-transfected CHO Cells, Neuronal RN46A Cells, and Human Platelets. To evaluate whether p38 MAPK-dependent SERT up-regulation is unique to SERTs or might extend to other biogenic-amine transporters, the inventors transfected CHO cells with human variants of SERT, NET, or DAT and tested for anisomycin-modulated uptake. They also included mouse SERT in these assays to monitor possible species selectivity of SERT modulation. As with rat SERT expressed by RBL-2H3 cells, the inventors found that anisomycin triggered an SB203580-sensitive elevation in 5-HT uptake by hSERT as well as by mSERT (FIG. 12). Non-transfected CHO cells expressed no paroxetine-sensitive 5-HT uptake. These cells also exhibited no additional 5-HT uptake after anisomycin treatment (data not shown). Interestingly, human NET activity was stimulated to a comparable degree as hSERT, with SB203580 also blocking stimulation back to basal levels. In contrast, DA transport activity induced by hDAT transfection displayed a small, but significant SB203580-sensitive inhibition of DA uptake. These findings argue against a global alteration in membrane potential or ion gradients in support of p38 MAPK-linked SERT modulation.

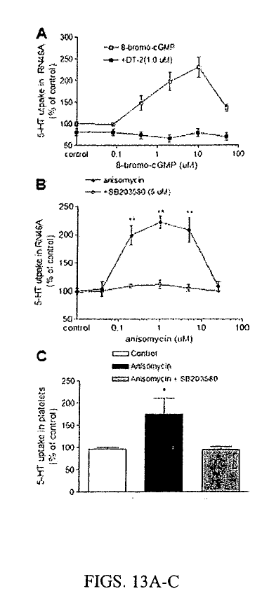

Next, the inventors sought evidence for a more general role of p38 MAPK in SERT modulation, examining anisomycin's effects in both a neuronal cell model and in platelets. The RN46A line is a serotonergic neuronal cell line, derived from rat E13 raphenucleus, that expresses native SERT (Eaton and Whittemore, 1996). As observed in RBL-2H3 cells, treatment with 8-bromo-cGMP induces a rapid, PKG antagonist (DT-2 (Dostmann et al., 2000))-sensitive increase in 5-HT transport (FIG. 13A). Anisomycin also dose-dependently stimulated 5-HT uptake in the RN46A cells, effects completely blocked by coincubation with SB203580 (FIG. 13B). Platelets are a readily accessible and well studied model featuring native SERT expression (Rudnick and Nelson, 1978). As found in nonneuronal and neuronal cell models, anisomycin also induced an SB203580-sensitive increase in 5-HT uptake in human platelets (FIG. 13C).

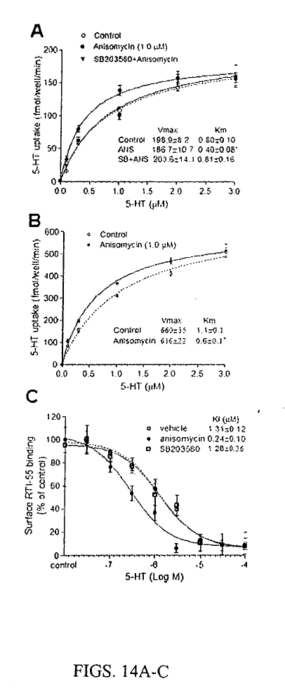

Anisomycin Treatment of RBL-2H₃Cells Selectively Reduces the 5-HT Transport Kₘ and Increases 5-HT Potency for Inhibition of Antagonist Binding—Our previous study revealed that p38 MAPK inhibition blocked AR-triggered increases in SERT activity but failed to attenuate elevations in surface SERT density (Zhu et al., 2004a,b). These remarkable findings suggested to us that p38 MAPK-mediated SERT stimulation might not arise from altered surface trafficking but rather represents a form of catalytic activation. The inventors took multiple approaches to explore this issue. First, they performed saturation kinetic analyses of 5-HT uptake in RN46A and RBL-2H3 cells, with or without anisomycin treatment. As shown in FIGS. 14A and 14B, anisomycin treatments significantly reduced the SERT Kₘ for 5-HT in RN46A cells (799±97 versus 396±80 nM, p<0.05) and in RBL-2H3 (1057±145 versus 628±70 nM, p<0.05) but effected no significant change in maximal transport capacity (Vₘₐₓ) in either model. Second, the inventors asked whether the reduction in 5-HT Kₘ reflected a change in 5-HT affinity. 5-HT does not bind to SERT with high enough affinity to determine its K_{D} value directly. However, 5-HT affinity can be estimated by deriving its apparent Kᵢ for displacement of a competitive antagonist.

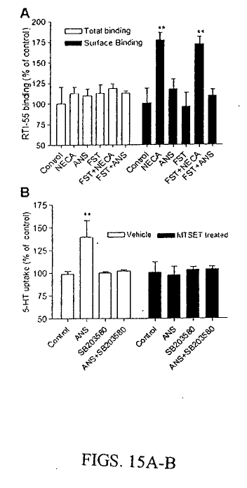

In studies examining 5-HT inhibition of whole cell [¹²⁵I]RTI-55 binding (FIG. 14C), the inventors observed that pretreatment of cells with 1 μM anisomycin for 10 min significantly reduced the apparent 5-HT Kᵢ for [¹²⁵I]RTI-55 competition (1.31±0.26 μM in the vehicle control, 0.24±0.005 μM in the presence of anisomycin, p<0.05). This effect was blocked by SB203580, which had no significant effect on 5-HT Kᵢ on its own. The studies noted above support the idea that p38 MAPKlinked SERT modulation arises not from surface trafficking but from catalytic modulation of preexisting surface-resident transporters. The inventors thus wished to evaluate directly possible changes in SERT total and surface density in the context of anisomycin stimulation. However, in the RBL-2H3 cell model, SERT protein levels do not permit biotinylation paradigms more suitable for heterologous expression systems. Instead, the inventors monitored the extent of 5-HT (surface)-displaceable versus paroxetine (total)-displaceable [¹²⁵I]RTI-55 binding to intact cells at 4° C., as previously described (Zhu et al., 2004a,b). Consistent with the results of saturation kinetic studies, anisomycin does not impact total surface-SERT binding (FIG. 15A). As a positive control that our assay can detect surface increases by agents known to elevate SERT density, the inventors demonstrate a significant increase in SERT surface density following NECA treatment (Zhu et al., 2004a,b). As expected for the acute nature of these treatments, total SERT (internal plus external pools), as defined by paroxetine-displaceable [¹²⁵I]RTI-55 binding, does not change in response to either anisomycin or NECA (FIG. 15A), indicating that changes in SERT synthesis/turnover do not have a role in this process.

Finally, if p38 MAPK activates surface SERTs (versus delivery of new carriers), then irreversible inactivation of the surface pool should eliminate anisomycin stimulation of 5-HT uptake. To test this possibility, the inventors treated cells with the membrane-impermeant cysteine-modifying reagent MTSET (Chen et al., 1997) to inactivate surface SERTs. Transport assays on control and MTSET-treated cells revealed that anisomycin-induced stimulation of SERT activity is abolished following the MTSET treatment (FIG. 15B), whereas similar treatments actually magnify the increases observed by stimuli (e.g., AR agonist NECA) that induce SERT exocytosis (Zhu et al., 2004a,b).

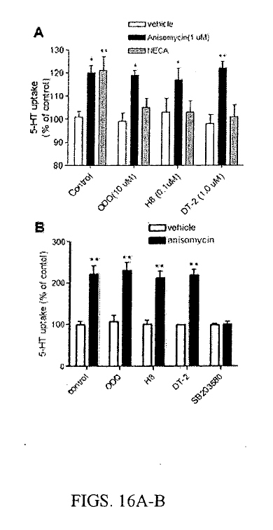

Inhibition of cGMP/PKG Does Not Impact Anisomycin-induced SERT Stimulation. To begin to evaluate targets of activated p38 MAPK-supporting SERT stimulation, the inventors next sought to verify formally that p38 MAPK lies downstream of cGMP production, a second messenger that augments 5-HT uptake in an SB203580-sensitive manner (Zhu et al., 2004a; Zhu et al., 2004b). The inventors therefore monitored the impact of the guanylyl cyclase inhibitor, ODQ, as well the PKG antagonists, H8 and DT-2, on anisomycin-stimulated 5-HT transport in RBL-2H3 and RN46A cells. Whereas LY83583 and ODQ completely abolished AR stimulation of SERT in RBL-2H3 cells (and in co-transfected models) (Zhu et al., 2004a,b), neither agent attenuated anisomycin stimulation of SERT activity in these models (FIGS. 16A and 16B). Additionally, both DT-2 and H8 blocked 8-Br-cGMP stimulation of SERT (data not shown) but failed to attenuate anisomycin's effects on SERT. Since AR stimulation triggers p38 MAPK activation (see FIG. 10), a lack of effect of ODQ, H8, and DT-2 on SB203580-sensitive, anisomycin-stimulated 5-HT uptake places p38 MAPK downstream of guanylyl cyclase and PKG in the SERT regulatory pathway. Moreover, both RBL-2H3 and RN46A cells appear to utilize these pathways (although with different quantifying effects) to regulate SERT activity.

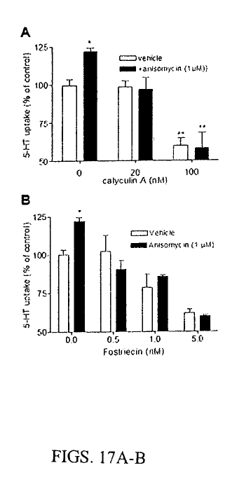

Catalytically Active PP2A Is Required for p38 MAPK-mediated SERT Stimulation. One can envision multiple mechanisms by which p38 MAPK activation modulates SERT activity, including a direct action on SERT as well as interactions with a growing list of SERT-associated proteins (Haase et al., 2001; Bauman et al., 2000; Carneiro et al., 2002; Horschitz et al., 2003). The inventors were particularly interested in PP2A, since the phosphatase is known to be activated by p38 MAPK (Westermarck et al., 2001; Liu and Hofmann, 2003), and the enzyme associates with surface SERT in a phorbol ester-, okadaic acid-, and 5-HT-sensitive manner (Bauman et al., 2000). They found (FIG. 17A) that the protein phosphatase 1/2A inhibitor, calyculin A, completely blocks anisomycin stimulation of SERT in RBL-2H3 cells at a concentration (20 nM) that fails to diminish basal 5-HT-uptake. At higher concentrations (100 nM), calyculin A also inhibits both basal and anisomycin-stimulated 5-HT uptake suggesting additional activities, most likely on SERT trafficking.

The inventors observed similar results with fostriecin (5 nM), an eve n more potent and selective PP2A inhibitor (FIG. 17B), supporting an essential role of catalytically activated PP2A in p38 MAPK stimulation of 5-HT uptake. Importantly, fostriecin, like SB203580, fails to block the NECA-induced enhancement of SERT surface density at a concentration that completely blocks anisomycin stimulation of 5-HT uptake (FIG. 15A). These findings suggest that, with respect to acute SERT up-regulation, PP2A is required for SERT catalytic activation but not for SERT plasma membrane insertion.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,659,774
U.S. Pat. No. 4,683,195
U.S. Pat. No. 4,683,202
U.S. Pat. No. 4,684,611
U.S. Pat. No. 4,800,159
U.S. Pat. No. 4,816,571
U.S. Pat. No. 4,873,191
U.S. Pat. No. 4,883,750
U.S. Pat. No. 4,952,500
U.S. Pat. No. 4,959,463
U.S. Pat. No. 5,141,813
U.S. Pat. No. 5,175,384
U.S. Pat. No. 5,175,385
U.S. Pat. No. 5,242,974
U.S. Pat. No. 5,264,566
U.S. Pat. No. 5,302,523
U.S. Pat. No. 5,312,734
U.S. Pat. No. 5,312,734
U.S. Pat. No. 5,384,253,
U.S. Pat. No. 5,384,261
U.S. Pat. No. 5,405,783
U.S. Pat. No. 5,412,087
U.S. Pat. No. 5,418,162
U.S. Pat. No. 5,418,162
U.S. Pat. No. 5,424,185
U.S. Pat. No. 5,424,185
U.S. Pat. No. 5,424,186
U.S. Pat. No. 5,428,148
U.S. Pat. No. 5,429,807
U.S. Pat. No. 5,436,327
U.S. Pat. No. 5,445,934
U.S. Pat. No. 5,464,765,
U.S. Pat. No. 5,472,672
U.S. Pat. No. 5,527,681
U.S. Pat. No. 5,529,756
U.S. Pat. No. 5,530,179
U.S. Pat. No. 5,532,128
U.S. Pat. No. 5,545,531
U.S. Pat. No. 5,554,501
U.S. Pat. No. 5,554,744
U.S. Pat. No. 5,556,752
U.S. Pat. No. 5,561,071
U.S. Pat. No. 5,565,186
U.S. Pat. No. 5,571,639
U.S. Pat. No. 5,574,146
U.S. Pat. No. 5,580,859
U.S. Pat. No. 5,589,466
U.S. Pat. No. 5,593,839
U.S. Pat. No. 5,599,695
U.S. Pat. No. 5,602,244
U.S. Pat. No. 5,612,486
U.S. Pat. No. 5,624,711
U.S. Pat. No. 5,625,125
U.S. Pat. No. 5,639,457
U.S. Pat. No. 5,656,610
U.S. Pat. No. 5,658,734
U.S. Pat. No. 5,700,637
U.S. Pat. No. 5,702,932
U.S. Pat. No. 5,736,524
U.S. Pat. No. 5,780,448
U.S. Pat. No. 5,789,215
U.S. Pat. No. 5,840,873
U.S. Pat. No. 5,843,640
U.S. Pat. No. 5,843,651
U.S. Pat. No. 5,843,663
U.S. Pat. No. 5,846,708
U.S. Pat. No. 5,846,717
U.S. Pat. No. 5,846,726
U.S. Pat. No. 5,846,729
U.S. Pat. No. 5,849,481
U.S. Pat. No. 5,849,486
U.S. Pat. No. 5,849,487
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,861,244
U.S. Pat. No. 5,863,732
U.S. Pat. No. 5,863,753
U.S. Pat. No. 5,866,331
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,919,626
U.S. Pat. No. 5,919,630
U.S. Pat. No. 5,925,517
U.S. Pat. No. 5,925,565
U.S. Pat. No. 5,928,862
U.S. Pat. No. 5,928,869
U.S. Pat. No. 5,929,227
U.S. Pat. No. 5,932,413
U.S. Pat. No. 5,935,791
U.S. Pat. No. 5,935,819
U.S. Pat. No. 5,945,100
U.S. Pat. No. 5,981,274
U.S. Pat. No. 5,994,624
U.S. Pat. No. 6,004,755
U.S. Ser. 10/407,846
Ansorge et al., Science, 306:879-81, 2004.
Ausubel et al., In: Current Protocols in Molecular Biology, John, Wiley & Sons, Inc, New York, 19946.
Baichwal and Sugden, In: Gene Transfer, Kucherlapati (Ed.), NY, Plenum Press, 117-148, 1986.
Bauman et al., J. Neurosci., 20:7571-7578, 2000.
Beaucage, Methods Mol. Biol., 20:33-61, 1993.
Belzung et al., Psychopharmacology (Berl). 97(3):388-91, 1989.
Blackman et al., Biophys. J., 71(1):194-208, 1996.
Bradley and Blakely, J. Neurochem., 69:1356-1367, 1997.
Bradley et al., Nature, 309:255-258, 1984.
Briley and Moret, Trends Pharmacol Sci., 14(11):396-397, 1993.
Brinster et al., Proc. Natl. Acad. Sci. USA, 82(13):4438-4442, 1985.
Bucknall et al., J. Am. Soc. Mass Spectrom., 13(9):1015-1027, 2002.
Carbonelli et al., FEMS Microbiol. Lett., 177(1):75-82, 1999.
Cargill, et al., Nat. Genet., 22:231-238, 1999.
Carneiro et al., J Neuroscience, 22:7045-7054, 2002.
Caspi et al., Science, 301:386-9, 2003.
Chandler et al., Proc. Natl. Acad. Sci. USA, 94(8):3596-601, 1997.
Chen and Okayama, Mol. Cell. Biol., 7(8):2745-2752, 1987.
Chen et al., J. Biol. Chem., 273:12675-12681, 1998.
Chen et al., J. Biolog. Chem., 272:28321-28327, 1997.
Chen et al., Nat. Biotechnol., 19:537-542, 2001.
Ciaranello, N. Engl. J. Med., 307:181-183, 1982.
Cocea, Biotechniques, 23(5):814-816, 1997.
Cohen et al., Science, 305:869-72, 2004.
Cook and Leventhal, Curr. Opin. Pediatrics, 8:348-354, 1996.
Coupar et al., Gene, 68:1-10, 1988.
Di Bella et al., Am. J. Med. Genet., 67:541-545, 1996.
Dostmann et al., Proc. Natl. Acad. Sci. USA, 97(26):14772-14777, 2000.
Eaton and Whittemore, Exp. Neurol., 140(2):105-114, 1996.
Edwards, Ann. Neurol., 34(5):638-645, 1993.
European Appln. 320 308
Evans et al., Nature, 292:154-156, 1981.
Fechheimer, et al., Proc Natl. Acad. Sci. USA, 84:8463-8467, 1987.
Fozzard, In: Peripheral actions of 5-hydroxytryptamine, Oxford University Press, New York, 1989.
Fraley et al., Proc. Natl. Acad. Sci. USA, 76:3348-3352, 1979.
Friedmann, Science, 244:1275-1281, 1989.
Galli et al., Proc. Natl. Acad. Sci. USA, 95(22):13260-13265, 1998.
Gershon, Aliment Pharmacol. Ther:, 13(2): 15-30, 1999.
Gillam et al., J. Biol. Chem., 253:2532-2539, 1978.
Giros et al., Mol .Pharmacol., 42(3):383-390, 1992.
Giros et al., Nature, 379(6566):606-612, 1996.
Glatt et al., Nat. Genet., 27:435, 2001
Glatt et al., Nat. Genet., 27:435-438, 2001.
Gobom et al., Anal. Chem., 72(14):3320-3326, 2000.
Gopal, Mol. Cell. Biol., 5:1188-1190, 1985.
Gossler et al., Proc. Natl. Acad. Sci. USA 83: 9065-9069, 1986.
Graham and Van Der Eb, Virology, 52:456-467, 1973.
Gu et al., J. Biol. Chem., 269:7124-7130, 1994.
Haase et al., Biochem. Soc. Trans., 29:722-8, 2001.
Hahn an d Blakely, Pharmacogenomics J., 2:217-35, 2002.
Harland and Weintraub, J. Cell Biol., 101(3):1094-1099, 1985.
Hastrup et al., Proc. Natl. Acad. Sci. USA, 98:10055-10060, 2001.
Hermonat and Muzycska, Proc. Natl. Acad. Sci. USA, 81:6466-6470, 1984.
Hoffman et al., Science, 254:579-580, 1991.
Hogan et al., In: Manipulating the Mouse Embryo: A Laboratory Manual, 2nd ed., Cold Spring Harbor Laboratory Press, 1994.
Horschitz et al., J. Neurochem., 86(4):958-965, 2003.
Horwich et al. J. Virol., 64:642-650, 1990.
IMGSAC, Am. J. Hum. Genet., 69:570-581, 2001.
Innis, et al., In: PCR Protocols. A guide to Methods and Application, Academic Press, Inc. San Diego, 1990.
Insel et al., Annals New York Academy of Sciences, 574-586, 1990.
Itakura et al., J. Am. Chem. Soc., 97(25):7327-7332, 1975.
Jacobs and Azmitia, Physiological Rev., 72:165-229, 1992.
Jaenich, Proc. Natl. Acad. Sci. USA, 73(4):1260-1264, 1976.
Jaenisch, Science 240:1468-1474, 1988.
Jahner et al., Nature 298:623-628, 1982.
Jahner et al., Proc. Natl. Acad. Sci. USA, 82:6927-6931, 1985.
Jayanthi et al., Mol. Pharmacol., 67:2077-87, 2005.
Jess et al., Biochem. Biophys. Res. Commun., 294:272-9, 2002.
Kaeppler et al., Plant Cell Reports, 9:415-418, 1990.
Kaneda et al., Science, 243:375-378, 1989.
Kato et al, J. Biol. Chem., 266:3361-3364, 1991.
Khorana, Science, 203(4381):614-625, 1979.
Kilic et al., Mol. Pharmacol., 64:440-6, 2003.
Launay et al., Am. J. Physiol., 266:526-536, 1994.
Lebrand et al., J. Comp. Neurol., 401:506-24, 1998.
Lesch et al., Science, 274:1527-1531, 1996.
Levenson et al., Hum. Gene Ther., 9(8):1233-1236, 1998.
Liu and Hofmann, Am. J. Physiol. Heart Circ. Physiol., 285(1):H97-103, 2003.
Macejak and Sarnow, Nature, 353:90-94, 1991.
MacKenzie and Quinn, Proc. Natl. Acad. Sci. USA, 96:15251-15255, 1999.
Maniatis, et al., Molecular Cloning, A Laboratory Manual, Cold Spring Harbor Press, Cold Spring Harbor, N.Y., 1990.
Manipulating the Mouse Embryo, Hogan eds. (Cold Spring Harbor Laboratory Press, Cold Spring Harbor, 1986
McCauley et al., Am. J. Med. Genetics, 127B: 104-112, 2004.
Melikian, Pharmacol. Ther., 104:17-27, 2004.
Melikian et al., J. Biol. Chem. 269:12290-12297, 1994.
Melikian et al., Mol. Pharmacol. 50(2):266-76, 1996.
Meltzer et al., Arch. Gen. Psychiatry, 38(12):1322-1326, 1981.
Meltzer, Annals New York Academy of Sciences, 486-499, 1990.
Miller and Hoffman, J.f Biolog. Chem., 269(44):27351-27356, 1994.
Miner et al., J. Comp. Neurol., 427:220-234, 2000.
Mirgorodskaya et al., Rapid Commun. Mass Spectrom., 14(14):1226-1232, 2000.
Moriguchi et al., J. Biol. Chem., 271(43):26981-26988, 1996.
Murphy et al., Mol. Interv., 4:109-23, 2004.
New et al., EMBO J., 17(12):3372-3384, 1998.
Nicolas and Rubinstein, In: Vectors. A survey of molecular cloning vectors and their uses, Rodriguez and Denhardt, eds., Stoneham: Butterworth, 494-513, 1988.
Nicolau and Sene, Biochim. Biophys. Acta, 721:185-190, 1982.
Nicolau et al., Methods Enzymol., 149:157-176, 1987.
Norregaard et al., EMBO J, 17:4266-4273, 1998.
Omirulleh et al., Plant Mol. Biol., 21(3):415-28, 1993.
Ozaki et al., Mol. Psychiatry, 8:895, 933-6, 2003.
Ozsarac et al., J. Neurochem., 82:336-44, 2002.
Pacholczyk et al., Nature, 350(6316):350-354, 1991.
Paul et al., Arch. Gen. Psychiatry, 38(12):1315-1317, 1981.
PCT Appln. WO 84/03564
PCT Appln. WO 90/07641
Pelletier and Sonenberg, Nature, 334(6180):320-325, 1988.
Pellow et al., J. Pharm. Pharmacol., 37(8):560-563, 1985.
Perry et al., Br. J. Psychiatry, 142:188-192, 1983.
Persico et al., J. Neurosci., 21:6862-73, 2001.
Piven et al., J. Autism Dev. Disord., 21:51-59, 1991.
Porsolt et al., Nature, 266(5604):730-732, 1977.
Potrykus et al., Mol. Gen. Genet., 199(2):169-177, 1985.
Potter et al., Proc. Natl. Acad. Sci. USA, 81:7161-7165, 1984.
Pritchard, Am. J. Hum. Genet., 69:124-37, 2001.
Qian et al., J. Neurosci., 17:45-47, 1997.
Qian et al., J. Neurosci., 15:1261-1274, 1995
Ramamoorthy et al., J. Biol. Chem., 273:2458-2466, 1998.
Ramamoorthy et al., Proc. Natl. Acad. Sci. USA, 90:2542-2546, 1993.
Ramamoorthy et al., Methods Enzymol. 296:347-70, 1998.
Ramamoorthy and Blakely, Science, 285:763-766, 1999.
Remington's Pharmaceutical Sciences, 15^{th} ed., pages 1035-1038 and 1570-1580, Mack Publishing Company, Easton, Pa., 1980.
Ridgeway, In: Vectors. A survey of molecular cloning vectors and their uses, Stoneham: Butterworth, pp. 467-492, 1988.
Rippe, et al., Mol. Cell. Biol., 10:689-695, 1990.
Robertson et al., Nature 322:445-448, 1986
Sambrook et al., (ed.), Molecular Cloning, Cold Spring Harbor Laboratory Press, Cold Spring Harbor, N.Y., 1989.
Sambrook et al., In: Molecular cloning, Cold Spring Harbor Laboratory Press, Cold Spring Harbor, N.Y., 2001.
Samuvel et al., J. Neurosci., 5:29-41, 2005.
Shannon et al., N. Engl. J. Med., 342:541-549, 2000.
Shu et al., Proc. Natl. Acad. Sci. USA, 100:5902-7, 2003.
Stewart et al., EMBO J. 6:383-388, 1987.
Stone et al., Am. J. Hum. Genet., 75:1117-1123, 2004.
Suranyi-Cadotte et al., Life Sci., 36(8):795-799, 1985.
Tate and Blakely, J. Biol. Chem., 269(42):26303-26310, 1994.
Temin, In: Gene Transfer, Kucherlapati (Ed.), NY, Plenum Press, 149-188, 1986.
Tur-Kaspa et al., Mol. Cell. Biol., 6:716-718, 1986.
Van der Putten et al., Proc. Natl. Acad. Sci. USA 82:6148-6152, 1985.
Wada et al., Nucleic Acids Res. 18:2367-2411, 1990.
Westermarck et al., Mol. Cell. Biol., 21(7):2373-2383, 2001.
Wong et al., Gene, 10:87-94, 1980.
Wu and Wu, Biochemistry, 27:887-892, 1988.
Wu and Wu, J. Biol. Chem., 262:4429-4432, 1987.
Wu et al., Biochim. Biophys. Acta, 1466:315-327, 2000.
Wu et al., Proc. Natl. Acad. Sci. USA, 86(8):2757-2760, 1989.
Yonan et al., Am. J. Hum. Genet., 73:886-897, 203.
Zhang et al., J. Biol. Chem., 272(20):13397-13402, 1997.
Zhong et al., Clin. Chem. ACTA., 313:147, 2001.
Zhu et al., J. Vasc. Res., 40(2): 140-148, 2003.
Zhu et al., J. Biol. Chem., 280(16):15649-15658, 2005.
Zhu et al., Mol. Pharmacol., 65:1462-74, 2004a.
Zhu et al., Eur. J. Pharmacol., 504:1-6, 2004b.

What is claimed is:

1. A method of modulating anti-depressent biogenic amine transporter (BAT) function comprising contacting a BAT-expressing cell with a modulator of the p38 mitogen-activated protein kinase (MAPK) pathway and at least one other BAT modulator.

2. The method of claim 1, wherein the BAT is the serotonin transporter (SERT) or the norepinephrine transporter (NET).

3. The method of claim 1, wherein the other modulator is a peptide, a polypeptide, a nucleic acid or an organopharmaceutical.

4. The method of claim 2, wherein said SERT modulator is a SERT enhancer.

5. The method of claim 2, wherein said SERT modulator is a SERT inhibitor.

6. The method of claim 2, wherein said NET modulator is a NET enhancer.

7. The method of claim 2, wherein said NET modulator is a NET inhibitor.

8. The method of claim 1, wherein said p38 MAPK modulator is an agonist.

9. The method of claim 1, wherein said p38 MAPK modulator is an antagonist.

10. The method of claim 1, wherein said p38 MAPK modulator is a PPA2, PKG or PDE-5 modulator.

11. The method of claim 1, wherein said BAT-expressing cell is a primary neuronal cell.

12. The method of claim 1, wherein said BAT-expressing cell is an immortalized neuronal cell.

13. The method of claim 1, wherein said BAT-expressing cell is recombinantly engineered to express a BAT.

14. A method of modulating biogenic amine transporter (BAT) function in a subject comprising administering to said subject a modulator of the p38 mitogen-activated protein kinase (MAPK) pathway.

15. The method of claim 14, wherein said cell is further contacted with a BAT modulator.

16. The method of claim 14, wherein said p38 MAPK modulator is an agonist.

17. The method of claim 14, wherein said p38 MAPK modulator is an antagonist.

18. The method of claim 14, wherein said p38 MAPK modulator is a PPA2, PKG or PDE-5 modulator.

19. The method of claim 14, wherein said BAT is the serotonin transporter (SERT) or the norepinephrine transporter (NET).

20. The method of claim 19, wherein said SERT modulator is an agonist.

21. The method of claim 19, wherein said SERT modulator is an antagonist.

22. The method of claim 19, wherein said NET modulator is an agonist.

23. The method of claim 19, wherein said NET modulator is an antagonist.

24. The method of claim 14, wherein said subject suffers from anxiety, a stress disorder, depression, autism, anorexia, alcoholism, hypertension, irritable bowel syndrome, schizophrenia, obsessive compulsive disorder, primary pulmonary hypertension, migraine, autism, or premenstrual syndrome.

25. A method of assessing drug sensitivity in a subject comprising determining the structure of a serotonin transporter in (SERT) cells of said subject.

26. The method of claim 25, wherein determining the structure comprises one or more of sequencing, Southern blot, Northern blot, Western blot, SNP analysis, RFLP, or primer extension.

27. The method of claim 25, wherein said subject suffers from anxiety, a stress disorder, depression, autism, anorexia, alcoholism, hypertension, irritable bowel syndrome, schizophrenia, obsessive compulsive disorder, primary pulmonary hypertension, migraine, autism, or premenstrual syndrome.

28. The method of claim 25, wherein determining the structure comprises one or more of: determining SERT protein mass, determining SERT phosphorylation state, determining SERT glycosylation state, or determining SERT 5HT flux/surface density ratios.

29. The method of claim 25, wherein determining comprises assessing for one or more of the following amino acid substitutions Thr4Ala, Gly56Ala, Glu215Lys, Lys605Asn, Pro612Ser, and Ile425Val.

30. The method of claim 25, further comprising making a drug selection or drug dosing decision based on the structure of the SERT, and optionally, administering a selected drug to said subject.

31. The method of claim 30, wherein said drug is a PKG or PDE-5 modulator.

32. The method of claim 30, wherein said drug is a p38 mitogen-activated protein kinase pathway (MAPK) modulator.

33. The method of claim 32, wherein the p38 mitogen-activated protein kinase pathway (MAPK) modulator is a PPA2 modulator.

34. The method of claim 32, wherein said p38 MAPK modulator is an agonist.

35. The method of claim 32, wherein said p38 MAPK modulator is an antagonist.

36. The method of claim 30, wherein said drug is a SERT modulator.

37. The method of claim 36, wherein said SERT modulator is an enhancer.

38. The method of claim 36, wherein said SERT modulator is an inhibitor.


SEQUENCE LISTING (4 sequences)

SEQ ID NO: 1 - DNA, 2508 bp, Homo sapiens
caaatccaagcacccagagatcaattgggatccttggcagatggacatcagtgtcatttactaaccagcaggatggagacgacgcccttgaattctcagaagcagctatcagcgtgtgaagatggagaagattgtcaggaaaacggagttctacagaaggttgttcccaccccaggggacaaagtggagtccgggcaaatatccaatgggtactcagcagttccaagtcctggtgcgggagatgacacacggcactctatcccagcgaccaccaccaccctagtggctgagcttcatcaaggggaacgggagacctggggcaagaaggtggatttccttctctcagtgattggctatgctgtggacctgggcaatgtctggcgcttcccctacatatgttaccagaatggagggggggcattcctcctcccctacaccatcatggccatttttgggggaatcccgctcttttacatggagctcgcactgggacagtaccaccgaaatggatgcatttcaatatggaggaaaatctgcccgattttcaaagggattggttatgccatctgcatcattgccttttacattgcttcctactacaacaccatcatggcctgggcgctatactacctcatctcctccttcacggaccagctgccctggaccagctgcaagaactcctggaacactggcaactgcaccaattacttctccgaggacaacatcacctggaccctccattccacgtcccctgctgaagaattttacacgcgccacgtcctgcagatccaccggtctaaggggctccaggacctggggggcatcagctggcagctggccctctgcatcatgctgatcttcactgttatctacttcagcatctggaaaggcgtcaagacctctggcaaggtggtgtgggtgacagccaccttcccttatatcatcctttctgtcctgctggtgaggggtgccaccctccctggagcctggaggggtgttctcttctacttgaaacccaattggcagaaactcctggagacaggggtgtggatagatgcagccgctcagatcttcttctctcttggtccgggctttggggtcctgctggcttttgctagctacaacaagttcaacaacaactgctaccaagatgccctggtgaccagcgtggtgaactgcatgacgagcttcgtttcgggatttgtcatcttcacagtgctcggttacatggctgagatgaggaatgaagatgtgtctgaggtggccaaagacgcaggtcccagcctcctcttcatcacgtatgcagaagcgatagccaacatgccagcgtccactttctttgccatcatcttctttctgatgttaatcacgctgggcttggacagcacgtttgcaggcttggagggggtgatcacggctgtgctggatgagttcccacacgtctgggccaagcgccgggagcggttcgtgctcgccgtggtcatcacctgcttctttggatccctggtcaccctgacttttggaggggcctacgtggtgaagctgctggaggagtatgccacggggcccgcagtgctcactgtcgcgctgatcgaagcagtcgctgtgtcttggttctatggcatcactcagttctgcagggacgtgaaggaaatgctcggcttcagcccggggtggttctggaggatctgctgggtggccatcagccctctgtttctcctgttcatcatttgcagttttctgatgagcccgccacaactacgacttttccaatataattatccttactggagtatcatcttgggttactgcataggaacctcatctttcatttgcatccccacatatatagcttatcggttgatcatcactccagggacatttaaagagcgtattattaaaagtattaccccagaaacaccaacagaaattccttgtggggacatccgcttgaatgctgtgtaacacactcaccgagaggaaaaaggcttctccacaacctcctcctccagttctgatgaggcacgcctgccttctcccctccaagtgaatgagtttccagctaagcctgatgatggaagggccttctccacagggacacagtctggtgcccagactcaaggcctccagccacttatttccatggattcccctggacatattcccatggtagactgtgacacagctgagctggcctattttggacgtgtgaggatgtggatggaggtgatgaaaaccaccctatcatcagttaggattaggtttagaatcaagtctgtgaaagtctcctgtatcatttcttggtatgatcattggtatctgatatctgtttgcttctaaaggtttcactgttcatgaatacgtaaactgcgtaggagagaacagggatgctatctcgctagccatatattttctgagtagcatatagaattttattgctggaatctactagaaccttctaatccatgtgctgctgtggcatcaggaaaggaagatgtaagaagctaa

SEQ ID NO: 2 - protein, 630 aa, Homo sapiens
METTPLNSQKQLSACEDGEDCQENGVLQKVVPTPGDKVESGQISNGYSAVPSPGAGDDTRHSIPATTTTLVAELHQGERETWGKKVDFLLSVIGYAVDLGNVWRFPYICYQNGGGAFLLPYTIMAIFGGIPLFYMELALGQYHRNGCISIWRKICPIFKGIGYAICIIAFYIASYYNTIMAWALYYLISSFTDQLPWTSCKNSWNTGNCTNYFSEDNITWTLHSTSPAEEFYTRHVLQIHRSKGLQDLGGISWQLALCIMLIFTVIYFSIWKGVKTSGKVVWVTATFPYIILSVLLVRGATLPGAWRGVLFYLKPNWQKLLETGVWIDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHVWAKRRERFVLAVVITCFFGSLVTLTFGGAYVVKLLEEYATGPAVLTVALIEAVAVSWFYGITQFCRDVKEMLGFSPGWFWRICWVAISPLFLLFIICSFLMSPPQLRLFQYNYPYWSIILGYCIGTSSFICIPTYIAYRLIITPGTFKERIIKSITPETPTEIPCGDIRLNAV

SEQ ID NO: 3 - DNA, 2576 bp, Mus musculus
catggagaccacacctttgaattctcagaaagtgctgtcagagtgtaaggacaaagaggactgccaagaaaatggtgttctgcagaagggtgtccccacaccagcagacaaggcagagcctggacaaatatccaatgggtactccgcagttcccagtacaagcgctggggatgaagcgccacactctacgccagctgccaccaccaccctggtggctgagattcaccaaggggaacgggagacctggggcaagaagatggatttcctcctgtctgtcattggctatgccgtggacctgggcaacatctggcgttttccctacatatgctaccagaatggtggaggggccttcctcctcccttacaccatcatggccatctttggggggatcccgctcttctacatggagctcgccctgggccagtaccaccgaaatgggtgcatttctatatggaggaagatctgcccgattttcaaaggcattggctatgccatctgcatcattgccttttatatcgcctcctactataacaccatcatagcctgggcgctctactacctcatctcctccttcacggaccagctgccctggaccagctgcaagaactcttggaacactggcaactgcaccaactacttcgcccaggacaacatcacctggacactccattccacgtcacctgctgaggagttttacttgcgccatgtcctgcagatccatcagtcaaagggactccaggacctggggaccatcagctggcagctggctctctgcatcatgctcatcttcaccattatctacttcagcatctggaaaggagtcaaaacgtctggcaaggtggtgtgggtgacagccaccttcccttacattgtcctttctgtcctgctggtgaggggagccacccttcctggagcctggagaggggttgtcttttacttgaaacccaactggcagaaactcttggagacaggggtgtgggttgatgctgcggctcagatctttttctctcttggcccggggtttggggttctcctggcgtttgctagctacaacaagttcaacaacaactgttaccaagatgccctggtgaccagtgtggtgaactgcatgacgagcttcgtctctggctttgtcatcttcacggtgcttggctacatggctgagatgaggaacgaagacgtgtccgaggtggccaaagacgcgggccccagcctccttttcatcacatatgcggaggcaatagctaacatgccagcatccacattctttgccatcatcttcttcctcatgttaatcacgctgggtttggatagcacgtttgcaggcctggaaggtgtgatcacagctgtgttggatgagtttcctcacatctgggccaagcgcagggaatggtttgtgctcatcgtggtcatcacttgcatcttgggatccctgctcacactgacatcaggaggggcgtatgtggtgaccctgctggaggagtacgccacggggccagcagtgctcactgtggctctcatcgaggccgtcgtcgtgtcttggttctatggaatcactcagttctgcagcgacgtgaaggaaatgctgggcttcagccccggatggttttggaggatctgctgggtggccatcagtcctctgtttctcctgttcatcatttgcagttttttgatgagtccaccccaactccggcttttccaatacaattatccccactggagtatcatcttgggctactgcataggaacatcgtctgtcatctgcatccctatatacatcatttatcggctgatcagcactccagggacacttaaggagcgcattattaaaagtatcactcctgaaacaccaacggaaattccgtgtggggacatccgcatgaatgctgtgtaacacactgagggagaggacatggcctcccagcccccgactcctcatctctgaaaagccccaccttgactcctcccctctaagccaagctgatgatgtaaggtctttctccatggagtcacagtcctaaagactatggtgcccagactcttgtgggttccaaccacttctttccatgaactctcttggacttactgccacattagctggtgacacggctgagctgacttggatatgtgaggagaggaaggaggggatgaacgccacccagtcatcagctagcttcaggtttagattaggtctgtgaacgtctgtatcatgttctgggtatgatcatattgccctgcatctgtttgcttctaaagccttcagtgttcatgaatacataaaccacctaagagaaaacagggatgtcttgctagccatatatattttctcagtagcatagaactctgtagccggaatctactagaaccctgtaacccacgtgctgctgtgaggttaagaaaggaagacgtaaagacgctacactgaaaactgatatatatgtgtgagctcttgtgtctgtccattgttgtctgtgtcccctcaattccaacactccgggcccattacaaactatataaatggcctctaatttttcttacattaaacagattctacctacaaaaaaaaaaaaaaaaaa

SEQ ID NO: 4 - protein, 630 aa, Mus musculus
METTPLNSQKVLSECKDKEDCQENGVLQKGVPTPADKAEPGQISNGYSAVPSTSAGDEAPHSTPAATTTLVAEIHQGERETWGKKMDFLLSVIGYAVDLGNIWRFPYICYQNGGGAFLLPYTIMAIFGGIPLFYMELALGQYHRNGCISIWRKICPIFKGIGYAICIIAFYIASYYNTIIAWALYYLISSFTDQLPWTSCKNSWNTGNCTNYFAQDNITWTLHSTSPAEEFYLRHVLQIHQSKGLQDLGTISWQLALCIMLIFTIIYFSIWKGVKTSGKVVWVTATFPYIVLSVLLVRGATLPGAWRGVVFYLKPNWQKLLETGVWVDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHIWAKRREWFVLIVVITCILGSLLTLTSGGAYVVTLLEEYATGPAVLTVALIEAVVVSWFYGITQFCSDVKEMLGFSPGWFWRICWVAISPLFLLFIICSFLMSPPQLRLFQYNYPHWSIILGYCIGTSSVICIPIYIIYRLISTPGTLKERIIKSITPETPTEIPCGDIRMNAV